(19) United States
(12) Patent Application Publication
(10) Pub. No.: US 2022/0386670 A1
(43) Pub. Date: Dec. 8, 2022

(54) SYSTEMS AND METHODS FOR MANUFACTURING API-CONTAINING EDIBLES, AND RESULTING EDIBLE PRODUCTS

(71) Applicant: Atomic Health, Inc., Dover, DE (US)

(72) Inventors: William Warren, Lafayette, CA (US); Walter Lee Smith, Danville, CA (US)

(21) Appl. No.: 17/833,297

(22) Filed: Jun. 6, 2022

Related U.S. Application Data
(60) Provisional application No. 63/197,189, filed on Jun. 4, 2021.
(60) Provisional application No. 63/328,306, filed on Apr. 7, 2022.

(51) Int. Cl. A23L 33/105; A23L 5/10; A23G 3/48; A21D 2/36; A61K 9/00; A61K 36/185

(52) U.S. Cl. CPC A23L 33/105; A23L 5/10; A23G 3/48; A21D 2/36; A61K 9/0056; A61K 36/185; A23V 2002/00

(57) ABSTRACT

This present disclosure provides reliable methods and apparatus for delivering a pharmaceutical active ingredient to a food product that is produced in a batch process. In one example, the active pharmaceutical ingredient is added after the batch food product has been cooked and cooled. The food product can be a candy, a baked good, or any alternative food product that is mixed as a batch and subsequently cooked and cooled.

### CROSS-REFERENCE TO RELATED APPLICATIONS

This claims priority to U.S. Patent Application Ser. No. 63/197,189 filed Jun. 4, 2021, and also claims priority to U.S. Patent Application Ser. No. 63/328,306 filed Apr. 7, 2022, the disclosure of each of which is hereby incorporated by reference as if set forth in its entirety herein.

### BACKGROUND

### Field of the Art

The disclosure relates to the field of cannabis products, and more particularly to the field of edible products comprising cannabis, and methods and systems for manufacturing the same.

### Discussion of the State of the Art

The legal cannabis industry is growing rapidly in the United States, Canada, and across the world. Conventionally, cannabis, or other active pharmaceutical ingredient (API) included in batch-produced cooked food products are typically added during the batch stage of production. In particular, as illustrated at FIG. 1, a method 100 for batch producing cooked food product includes a step 101 of providing ingredients of a food product to be prepared. For instance, the providing step can include the step 102 of mixing a plurality of edible ingredients together to produce the mixture of batch food product. In some examples, a quantity of the food product can be removed from the mixture to produce a desired quantity of batch food product. The batch food product is then cooked at step 103 for a desired duration of time at a desired temperature, and subsequently cooled at step 104. The batch food product is then apportioned at step 106 into individualized food products, and then packaged at step 108 as desired. In some examples, the individualized food products are subjected to a final cooling step 107 prior to packaging.

Conventional API-adding processes include adding the API to the batch food product, and performing one or more mixing operations in the attempts to obtain a homogenous mixture of the API. Thus, the final individualized food products contain a quantity of the API. However, the present inventors have discovered that the conventional dosing sequence can cause the produced food products to suffer from substantial variations in the dosage of API. As a result, the pharmaceutical effect that is experienced when consuming one of the individualized food products will substantially vary with respect to the pharmaceutical effect that is experienced when consuming another of the individualized food products. The difference can have a profound impact when the food product is bite-sized. When the food product is larger than bite-sized, the variations of API dosing can further cause a first region of the food product to include substantially more or less API than a second region of the food product. Thus, the pharmaceutical effect that is experienced when consuming the first region of the food product will substantially vary with respect to the pharmaceutical effect that is experienced when consuming the second region of the food product.

Therefore, what is needed is an improved batch-produced cooked food product having a dosed API that has greater consistency among the produced individualized food products than conventionally achieved.

### SUMMARY

In one example, a method of preparing a food product can include the steps of providing a mixture of batch food product, cooking the mixture of batch food product such that the food product defines a cooked batch food product, and cooling the cooked batch food product, such that the cooked batch food product defines a cooled food product. The method can further include, after the cooling step, the step of dosing the cooled food product with a cannabinoid.

### BRIEF DESCRIPTION OF THE DRAWINGS

The following detailed description will be better understood when read in conjunction with the appended drawings, in which there is shown in the drawings example embodiments for the purposes of illustration. It should be understood, however, that the present disclosure is not limited to the precise arrangements and instrumentalities shown. In the drawings:

FIG. 1 is a flow chart showing steps associated with a conventional method of batch preparing cooked food product;

FIG. 2A is a schematic perspective view of a system for delivering an active pharmaceutical ingredient to an edible product;

FIG. 2B is an enlarged schematic perspective view of a portion of the edible product illustrated in FIG. 2A;

FIG. 3A is a schematic view of a dosing zone of the system illustrated in FIG. 2A;

FIG. 3B is a perspective view of a microdroplet in one example;

FIG. 3C is a side elevation view of a microdroplet in another example;

FIG. 4 is a plan view of the edible product illustrated in FIG. 2A, showing a delivery zone;

FIG. 5A is a perspective view of mixed nuts dosed with an active pharmaceutical ingredient in accordance with aspects of the present disclosure;

FIG. 5B is a perspective view of dried fruit dosed with an active pharmaceutical ingredient in accordance with aspects of the present disclosure;

FIG. 5C is a perspective view of a baked good dosed with an active pharmaceutical ingredient in accordance with aspects of the present disclosure;

FIG. 5D is a side elevation view of a gummy candy dosed with an active pharmaceutical ingredient in accordance with aspects of the present disclosure;

FIG. 5E is a perspective view of a tongue depressor dosed with an active pharmaceutical ingredient in accordance with aspects of the present disclosure;

FIG. 6A is a perspective view of a dosing machine constructed in accordance with one example;

FIG. 6B is a schematic view of a method of dosing using the dosing machine illustrated in FIG. 6A;

FIG. 7 is a flow chart showing steps associated with a method of batch preparing cooked food product in accordance with one example;

FIG. 8 is a flow chart showing steps associated with a method of batch preparing cooked food product in accordance with another example;

FIG. 9A is a flow chart showing steps associated with a method of batch preparing cooked food product in accordance with still another example;

FIG. 9B is a schematic view of a mold cavity that receives food product in one example;

FIG. 10 is a flow chart showing steps associated with a method of batch preparing cooked food product in accordance with yet another example;

FIG. 11 is a flow chart showing steps associated with a method of batch preparing cooked food product in accordance with still another example; and

FIG. 12 is a schematic view of a mixing device in one aspect of the present disclosure.

### DETAILED DESCRIPTION

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

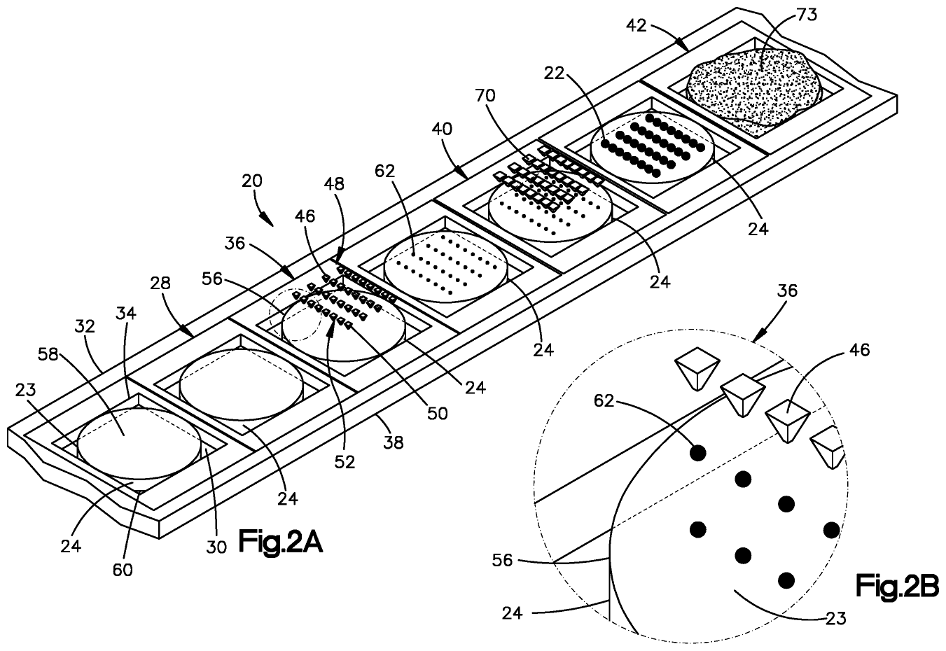

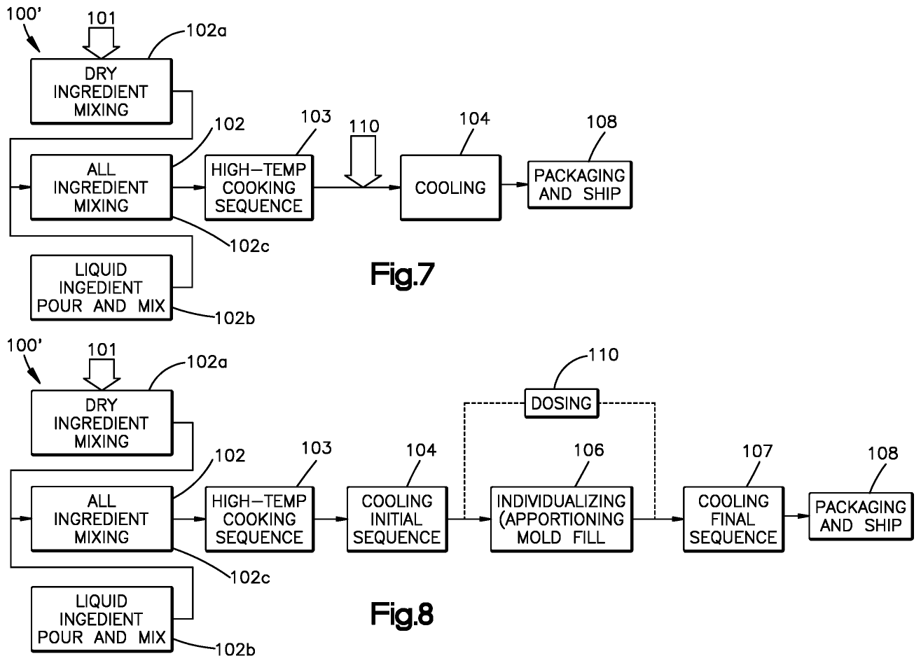

As described below with reference to FIGS. 7-11, methods are described for preparing food product with a desired dosage of API by dosing the API at one or more stages of the food preparation process to reduce opportunities for errors in the final dosage in the individualized food product. First, as will now be described with reference to FIGS. 2-6 generally, systems and methods are provided for manufacturing cannabis edibles, and resulting edible products

### Methods and Systems for Adding Cannabis to Edible Products

According to an aspect, methods for transforming an edible products or other substrates into a cannabis or other API-containing products are disclosed. The method can include the step of delivering an API such as a cannabinoid to the substrate, which can be an edible product or non-edible product. The delivering step can be performed with any suitable one or more applicators that deliver a predetermined dose of the cannabinoid. The term “cannabinoid” as used herein refers to any extract from a marijuana plant or hemp plant, such as CBD, THC, or any alternative cannabinoid, alone or in combination with any one or more of a flavonoid and a terpene. The extract can be in its pure form or processed as desired, including as example, emulsified forms of cannabinoid fluids. While this disclosure provides for the addition of at least one cannabinoid to a substrate, and thus cannabinoids are contemplated as a market for the final product, applications of the systems and methods disclosed herein are possible and envisioned that do not involve cannabis, including (but not limited to) other active pharmaceutical ingredients (API). For instance, applications of the systems and methods disclosed herein are possible and envisioned to include active pharmaceutical ingredients (APIs) including one or more cannabinoids, any alternative one or more over-the-counter (OTC) or prescription drugs including those that provide one or both of a health benefit or recreational drug experience, or otherwise controlled ingestible materials. Thus, reference herein to an active pharmaceutical ingredient can include any one the following: cannabis, one or more cannabinoids in either natural oily forms or emulsified forms, one or more over-the-counter drugs, one or more prescription drugs, one or more flavonoids, one or more terpenes, and any desired behavior modifying consumable. Thus, reference to one or more of the active pharmaceutical ingredients herein can apply equally to any other of the active pharmaceutical ingredients identified herein. According to an aspect of the present disclosure, a method of delivering a cannabinoid or conventional drug may also be used to deliver homeopathic remedies, herbal supplements with flavors or odors, and so forth, all of which are also APIs, to an edible product. The resulting edible product can be referred to as a “nutraceutical,” as its definition is “a food containing health-giving additives or having medicinal benefit. All APIs described herein can be provided as a natural or synthetic compound as desired.

The active pharmaceutical ingredient can be added to a substrate to produce an active-containing substrate, which can include edible food products, edible non-food products, or other inedible substrates. Edible food products can include, by way of example and not limitation, hard candy, chocolates brownies, cookies, soft candies such as gummy candy, savories such as trail mix bars or dried meat pieces, and the like. Thus, in some examples, the edible food products can be cooked food product. In some specific examples, the edible food products can be baked food product. food edible food product can be bite sized, such as M&M candy, gummy candies, chocolate kisses, or the like, or can be designed to require more than one bite for full consumption, such as a cookie. Thus, in some examples, the edible food product can include a plurality of mixed ingredients. As will be appreciated from the description below, the food product can be fully prepared prior to addition of the API to the food product. In other examples, the API can be added during preparation of the food product. In some examples, the food products can be a dehydrated food product, such as dried fruit or jerky. In other examples, the edible product can be freeze dried. In still other examples, the edible product can be a raw food product, such as nuts or fruit.

It will be appreciated that the application of the active pharmaceutical ingredient to the food product can allow for a broader range of food product to be made with active pharmaceutical ingredients. Further, the API can be more accurately dosed compared to conventional methods. When the active pharmaceutical ingredients are applied to previously prepared food product, APIs having short shelf lives can be applied to the substrate and ingested in a shorter period of time with respect to active pharmaceutical ingredients that are combined with the raw ingredients that are then processed to prepare the food product. When the API is added during the food preparation process, the API can be more accurately dosed compared to conventional methods whose API is included in bulk ingredients that are mixed prior to cooking or baking.

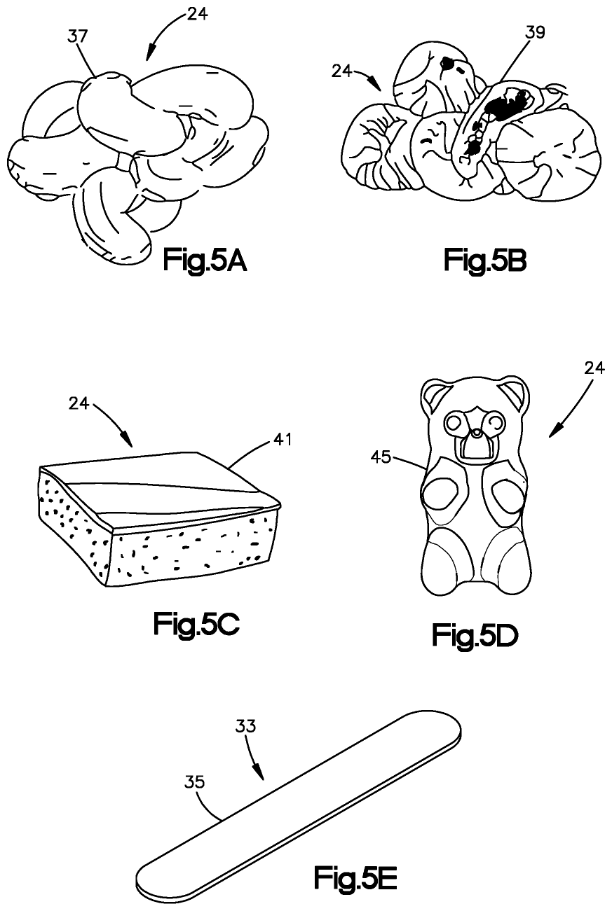

In still other examples, as described above, the substrate that is dosed with the API can be an edible non-food product. While the primary purposes of a food product is the delivery of nutrients, the primary purposes of non-food products is to deliver an API, either alone or with a carrier. Examples of edible non-food products can include a dissolvable material, such as a slip, or can be a capsule, pill, tablet, nugget, or the like. Alternatively still, as noted above, the substrate can be an inedible product 33. That is, the substrate is not designed for human consumption, but is designed to be placed into the mouth. One such nonlimiting example is a tongue depressor 35 (see FIG. 5E).

In one aspect, a plurality of active-containing substrates can be provided as a set, wherein some of the substrates have different dosages of the active pharmaceutical ingredients and are designed to be ingested at different times among a period of time, such as different days of the week. Thus, a desired dosage profile can be delivered to the patient throughout the period of time. Alternatively or additionally, one of the active-containing substrates can contain a different at least one active pharmaceutical ingredient. Thus, the set of active-containing substrates can be designed to be sequentially ingested (that is, ingested one after the other) over the period of time, thereby delivering a desired predetermined sequence of active pharmaceutical ingredients to the patient.

The active pharmaceutical ingredient, including one or more cannabinoids, can be delivered to the substrate in liquid form as an API-containing liquid or in granular solid form as an API-containing solid. The API-containing liquid can be in the form of a pure API, such as a resin, or can be in the form of a concentration of API in a liquid carrier such as a solvent. The API-containing solid can be in the form of pure API, such as a powder, or can be a mixture of the API with any other suitable material. API-containing liquids and API-containing powders to be delivered to a substrate can be referred to as API-containing material. In some examples, an applicator can deliver microquantity of the active pharmaceutical ingredient to the substrate. For instance, the microquantity can be carried by a solvent and delivered by the applicator as microdroplets each having a volume in a range from approximately 2 nanoliters to approximately 10 microliters, such as from approximately 25 nanoliters to approximately 2 microliters. For instance, the microdroplets can have a volume that is in a range from approximately 25 nanoliters to approximately 1 microliter. The microdroplets can have a concentration of API as desired. For instance, the concentration of API can range from approximately 50 micrograms per microliter of solution to approximately 1 milligram per microliter of solution. In other examples, the liquid can be a pure resin of the API.

It is envisioned in one example that applicators delivering like dosages of API to like substrates can deliver approximately the same sized microdroplets to the like substrates. Thus, for instance when delivering the API to dried fruits and/or nuts designed to have the same dosage of API, the applicators can deliver the approximately same volume and number of microdroplets to each dried fruit and/or nut, or to a group, such as a serving, of dried fruits and/or nuts.

As will be described in more detail below, dosing heads can be provided that are configured to deliver microdroplets of any suitable volume, such as the volumes described above. Therefore, each microdroplet can contain a microquantity of API in a range from approximately 0.1 micrograms to approximately 10 milligrams, such as from approximately 1 milligram to approximately 2 milligrams. It is recognized, however, that the microdroplets can have any volume as desired. Further, it is recognized that each microdroplet can contain different quantities of API depending, for instance, on the volume of the microdroplet. The microquantity of API in a microdroplet can allow the dosage of API delivered to a substrate to be precisely controlled. For instance, the respective volumes of microdroplets can be delivered to a substrate within a range from approximately 1% to approximately 10%, for instance approximately 5%, from a target volume of microdroplets at three sigma. Thus, the dosage of API in each microdroplet can be delivered to the substrate within a range from approximately 1% to approximately 10%, for instance approximately 5%, from a target dosage at three sigma. The target volume of microdroplets can vary based on the surface area or volume of the substrate to which the microdroplets are to be applied. Similarly, the dosage of API to be delivered to a substrate can likewise be within a range from approximately 1% to approximately 10%, for instance approximately 5% at three sigma, from a target dosage of API to the substrate or a serving of packaged substrates, such as dried fruits or nuts. The target volume of microdroplets can vary based on the surface area or volume of the substrate to which the microdroplets are to be applied.

Thus, in one example, the microquantity can be delivered to a substrate in the form of successive individualized (e.g., one at a time) microdroplets. The use of microdroplets may help in precise dosage administration of the active pharmaceutical ingredient compared to conventional techniques. The microdroplets can be delivered to an outer surface of the substrate by a 3D printer, inkjet printer, or other suitable printing process. Alternatively or additionally, the microdroplets can be delivered to the outer surface of the substrate by precision spraying. Alternatively or additionally still, the microdroplets can be delivered to an internal location of the substrate that is surrounded by the outer surface. For instance, the microdroplets can be mechanically injected, or can be delivered with an air gun that shoots the microdroplets toward the substrate in a burst of high-pressure air (in an analogous way to how some vaccines and other drugs may be administered subcutaneously without injections with needles). Without being bound by theory, it is believed that the microdroplets to an internal location of the substrate may also assist in facilitating administration or ingestion of potentially bitter-tasting (or strongly cannabis-tasting) formulations by providing a means to add small amounts of such material to a much larger amount of edible product.

The microdroplets including the API can be delivered to the substrate. The microdroplets can include a solution that includes the at least one cannabinoid in its liquid form as the solute mixed with any suitable solvent. In order to assist in achieving predictable doses of the at least one cannabinoid, the at least one cannabinoid can be substantially homogeneously mixed with the solute. Alternatively, the microdroplets can consist of or consist essentially of the cannabinoid extract, either purified, partially purified, or unpurified, in liquid form having a desired viscosity that allows for the cannabinoid extract to be reliably dispensed. In some examples, the liquid can be heated to achieve the desired viscosity without mixing the cannabinoid extract in a solute. In some examples, the microdroplets can have an oily or hydrophilic nature. For instance, microdroplets may be multilayered, with a protein or other protective coating surrounding a precise dosage of an oil-based or water-based formulation. Because the concentration of the cannabinoid in the liquid can be known, a volume of liquid can be predetermined and delivered to the substrate to achieve a desired predetermined approximate dose of the cannabinoid. In some other examples, the microdroplets may be of an emulsified cannabinoid liquid.

In other aspects, the active pharmaceutical ingredient, which can include one or more cannabinoids, can be added to the substrate in a granular form. For instance, the cannabinoid extract, which can be purified, partially purified, or unpurified, can be delivered to the substrate as a powder. In some examples, the cannabinoid can be crystallized and ground to produce the powder. Because the concentration of the cannabinoid in the powder can be known, a mass of the powder can be predetermined and delivered to the substrate to achieve a desired dose of the cannabinoid.

The liquid or powder to be delivered to the substrate can include a single desired cannabinoid. Thus, the single desired cannabinoid can be delivered to the substrate. Alternatively, a plurality of different liquids or powders can be delivered to the substrate, each containing their own different one or more cannabinoids. Accordingly, by delivering multiple different liquids and powders, a plurality of desired cannabinoids can be delivered to the substrate. The liquids and powders can be delivered in the same quantity or in different quantities. Accordingly, the ratio of one or more cannabinoids relative to one or more other cannabinoids can be controlled. In other examples, the liquid or powder to be delivered to the substrate can include a plurality of cannabinoids, either in equal proportions or in desired ratios. Thus, a single liquid or powder can be delivered to the substrate to deliver either a single cannabinoid or a plurality of cannabinoids. The plurality of cannabinoids delivered to the food product with one or more powders or liquids can include greater than one cannabinoid up to the full range of cannabinoids, such as approximately 113 cannabinoids.

Because the cannabinoid is added to the edible product after the edible product has been prepared, it should be appreciated that the cannabinoid is not subjected to the food preparation process. Thus, the cannabinoid is not subjected to the mixing of ingredients of the food product, the cooking of the food product, the freeze drying of the food product, the dehydration of the food product, or the like. As a result, the active pharmaceutical ingredient is not subject to processes that might otherwise degrade the efficacy of the active pharmaceutical ingredient. The present disclosure recognizes, however, that the methods of delivering the cannabinoid to prepared food product can further be applied to raw food product, such as raw fruit and nuts, edible non-food product, and other non-edible substrates.

According to another aspect, an API such as cannabis or other formulations may be applied via any suitable printing process, including 3D, inkjet printing, or any suitable alternative printing process. In some cases, such printing can be used to apply, for example, any suitable label such as a cannabis warning symbol or warning, and the ink dots used in the printing could be composed largely of a targeted formulation derived from cannabis or hemp. In some aspects, formulations used for precise addition to edible products may comprise a substantial fraction of one or more of a plurality of any suitable cannabinoids. Non-limiting examples of such cannabinoids include THC, CBD, or of a combination of one or both of these and other cannabinoids. In some aspects, a mix of cannabinoids can be supplemented by one or more terpenes or flavonoids, which terpenes and/or flavonoids could be extracted from cannabis or hemp, or could be provided as pure substances acquired or synthesized commercially from other sources. Further, bitter or strong cannabis flavors may be hidden within the much more prevalent flavor of the “host” edible product. Alternatively or additionally, the at least one cannabinoid can be deposited on a location of a product that is not designed to be brought into direct initial contact with the tongue during ingestion, thereby further masking the taste of the at least one cannabinoid. For instance, the at least one cannabinoid can be applied to the top rounded surface of a cookie, it being recognized that cookies are designed to be placed into the mouth with the bottom flat surface against the tongue. Alternatively or additionally, the microdroplets can be coated with a sugar or other suitable taste-masking agent as desired.

In some aspects, soft edible products such as chocolate, gummies, licorice, and the like can be used as a “carrier” or “host” for a quantity of cannabinoid that can be injected into the soft edible product (by air gun, or by needle, or by other suitable methods known in the art), to push the added material into the bulk of the soft edible product. Cannabis flavors may be masked by such an approach. In some aspects, energy such as infrared light, forced air, or microwaves may be applied to a surface of an edible product to soften (or further soften) the material in a small area, and cannabis or hemp-derived material may be injected into the area pre-treated with infrared more easily (or to a great depth in the host edible product). The energy can be applied before injection, after injection, or both before and after injection. In other examples, the at least one cannabinoid can be applied to multiple surfaces of the edible product up to all surfaces of the edible product.

For edible products, for instance candies, hard or soft, or any other suitable edible substrate, a visible design with CBD, THC, or other cannabinoid formulations acting as the ink can be printed on the item. One may print microdots having an indication of the dosage of the cannabinoid formulation as desired. Doses may also be sprayed on, or added as an additional layer, such as a candy or chocolate layer. In some aspects, the at least one cannabinoid can be mixed in with food ingredients, particularly when such ingredients are suitable for addition, shortly prior to packaging or delivering a prepared edible product (for example, as an ingredient in an icing or other coating, or as part of the sugar coating applied to gummy candies.

In some aspects, an edible product can be coated with small oil spheres or a coating of solid powder, each containing the at least one cannabinoid, to block taste or hide cannabis flavor. In some aspects, colorant may be added to the at least one cannabinoid prior to delivering the at least one cannabinoid to the edible product, in order to blend the formulation blend with the coloring of the edible product.

According to an aspect, the applicator sprays the API onto a surface of the edible product; wherein the cannabis or hemp-derived material either diffuses into or remains on the surface of the cannabis or hemp-derived material.

According to an aspect, the applicator shoots the API-containing liquid into the edible product.

According to an aspect, the applicator squirts the API-containing liquid into the edible product.

According to an aspect, the applicator squirts the API-containing liquid onto a surface of the edible product.

According to an aspect, the applicator stamps API-containing material onto a surface of the edible product.

According to an aspect, the applicator prints API-containing material onto a surface of the edible product.

According to an aspect, the applicator sprinkles API-containing material onto a surface of the edible product.

According to an aspect, the applicator builds one or more API-containing micro-pills on a surface of the edible product.

According to an aspect, the applicator adds a conformal coating of the API-containing material to the edible product.

According to an aspect, the applicator encapsulates or mixes the API-containing material with one or more modifiers that are configured to modify at least one or more of flavor, mechanical properties, or aesthetics of applied cannabis or hemp material before adding to the edible product.

According to an aspect, the applicator adjusts size, location, or distribution of the API-containing material on the edible product to modify flavor or aesthetics.

According to an aspect, the applicator adjusts one or both of a concentration of the API in a solution or an ingredient of the solution.

According to an aspect, energy is added to a surface of the edible product to increase adhesion of the API-containing material to the edible product. According to an aspect, energy is added to increase a temperature at the surface. In one aspect, the temperature can be increased by directing at least one of forced air, microwaves, and light, such as infrared light, to the surface. The energy can be added prior to delivering the API-containing material to the edible product, after delivering the API-containing material to the edible product, or both before and after delivering the API-containing material to the edible product.

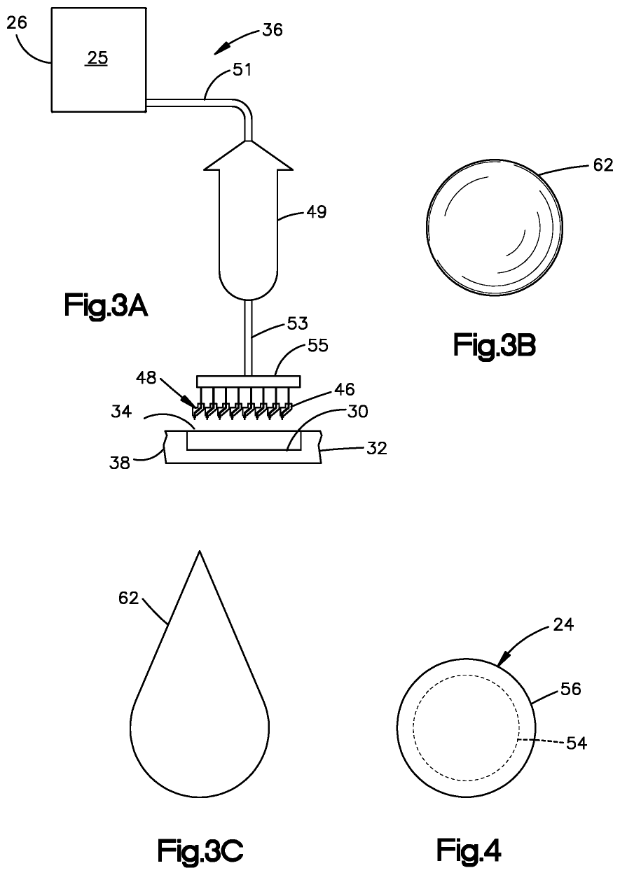

Referring now to FIGS. 2A-3, all of the above method steps and apparatus described herein, including an active-containing substrate, can be incorporated into or provided by any suitable system. While one such system 20 is illustrated and described herein, it is recognized that numerous alternatives are available for dispensing an approximate dose of the active pharmaceutical ingredient onto or into a desired substrate as described above. In one example, the system 20 is configured to deliver an active pharmaceutical ingredient 22 to a substrate 23, which can be configured as an edible product 24, thereby producing an active-containing substrate. When the substrate is an edible product, the active-containing substrate can be referred to as an active-containing edible product. As described above, the edible product 24 can be any suitable fully prepared food product. Alternatively, as is described in more detail below, the edible product 24 can be dosed with the API at one or more steps of a food production process that reduces opportunity for dosage inconsistencies that are common using conventional dosing techniques. Further, as described above, the active pharmaceutical ingredient can include at least one cannabinoid, at least one alternative drug or material that provides a health benefit or recreational drug experience, or any desired alternative ingestible controlled substance as designated by law. It will be appreciated that the system 20 can provide a cost-effective and efficient method for providing a product line of substrates having desired amounts and types of active pharmaceutical ingredients.

In some instances, it may be desirable to add one or more auxiliary edible products to the prepared edible product 24, either before or after delivering the active pharmaceutical ingredient 22 to the edible product 24. Examples include adding icing to a cookie, or frosting to a brownie or cake. However, in these examples, the cookie and brownie can have been fully cooked or otherwise prepared prior to adding the pharmaceutical ingredient. The system 20 can include one or more up to all of a delivery station 28 that is configured to receive one or more edible products, a dosing station 36 configured to deliver an approximate dose of the active pharmaceutical ingredient (API) 22 to the one or more edible products, a post-processing station 40, and a packaging station 42 that can be configured to package the edible product 24 that carries the approximate dose of the active pharmaceutical ingredient 22. In some examples the approximate dose can be a precise dose as described herein. The post-processing station 40 can be configured to at least one of 1) dries a solvent, for instance, when the API is delivered as a solution, 2) changes, for instance increases, a viscosity of the API, 3) further adheres the API to the substrate, 4) disperses the API along the substrate, and 5) increases absorption or diffusion of the API into the substrate. Operation of the system can be controlled by any suitable controller, such as the Champion 3700 Digital Dispensing Benchtop System commercially available from Creative Automation Company, having a place of business in Sun Valley, Calif.

The terms “substantial,” “approximate, “about,” and words of similar import when used with respect to a quantity, volume, mass, weight, dosage, size, shape, direction, or other parameter, include the stated parameter specifically along with ranges within plus and minus 20% of the stated parameter, for instance plus and minus 10% of the stated parameter, including within plus and minus 5% of the stated parameter, such as within plus and minus 2% of the stated parameter, including plus and minus 1% of the stated parameter.

When the at least one active pharmaceutical ingredient is to be delivered as an API-containing liquid 25, which can be a solution of the type described above or a pure API for instance as an oil, the system 20 can include a holding tank 26 that is configured to retain the liquid 25. Thus, while the liquid 25 can be a pure cannabis extract in one example, in other examples the cannabis extract can be mixed or otherwise combined with one or more other materials as desired, such as a solvent. In one example, the liquid is a solution having an approximate concentration of the cannabinoid or other active pharmaceutical ingredient 22 described above. The approximate concentration of the active pharmaceutical ingredient in the API containing material can be a known concentration described above. Thus, the active pharmaceutical ingredient 22 can define the solute of the solution, and the solution can define any suitable solvent. In one example, the solvent can be an alcohol, such as ethanol or any alternative alcohol as desired, or any other viscosity reducing agent as desired. In one example, the liquid 25 can contain a concentration of the active pharmaceutical ingredient that is in a range from approximately 40% to approximately 90%, such as from approximately 50% to approximately 70% by volume in solution with the solvent. It is recognized that the solvent will be removed substantially in its entirety during a subsequent drying step. For instance, the solvent can easily evaporate after being applied to the edible product 24. Nevertheless, it may be desired for the solvent to be safe for consumption in trace amounts.

Alternatively, the cannabinoid or other active API-containing liquid 25 can be a stand-alone extract, meaning that it is not mixed with a carrier that is designed to be burned or otherwise evaporated off. The extract can be purified, partially purified, or unpurified as desired. It is recognized that such stand-alone extracts can be in the form of a resin having a relatively high viscosity that can prevent the extract from being suitably free flowing for easy delivery to the substrate 23. Therefore, as described in more detail below, the system 20 can include one or more heaters that are configured to raise the temperature of the extract, thereby lowering the viscosity of the API-containing liquid. Alternatively or additionally, an additive such as alcohol can be added to the liquid 25 that lowers the viscosity of the liquid. The alcohol readily evaporates after the liquid 25 has been applied to the substrate 23. It is recognized that the extract having a suitably low viscosity can be readily delivered to the substrate in any manner described herein. For instance, it can be desirable to maintain the extract at a heated temperature during application of the extract to the substrate 23. The heated temperature can range from about 100 degrees F. to about 200 degrees F., such as at least 100 degrees F. or at least 150 degrees F. In one example, the heated temperature can be in a range from about 150 degrees F. to approximately 180 degrees F. Although it is envisioned that the extract has a sufficiently low viscosity at room temperature, it may nevertheless be desirable in some instances to maintain the solution at the heated temperature. Because the approximate dose of the at least one cannabinoid in the liquid 25 is known, a predetermined approximate volume of the liquid 25 delivered from the holding tank 26 to the dosing station 36, and thus to the edible idem 24, can contain approximately a predetermined approximate desired dose of the pharmaceutical ingredient 22.

The delivery station 28 can be configured to receive a plurality of substrates 23 such as a plurality of edible products 24. This, while the substrates are illustrated as edible products 24, it is recognized that the substrates can be configured as any suitable alternative substrate as described above. In one example, the system 20 includes one or more support surfaces 30 of at least one support member 32 that are configured to receive and support a respective one or more edible products 24. The support surfaces 30 can be defined by respective predetermined locations of the support member 32. The predetermined locations can be defined by geometric markings. Alternatively or additionally, the predetermined locations can be defined by pockets 34 that are defined by the support member 32. At least one or both of the support member 32 and the dosing station 36 can be movable so as to bring the dosing station 36 into alignment with the edible product 24. The dosing station 36 can be configured to deliver the approximate volume of the liquid 25 to one edible food product at a time, or can be configured to deliver a plurality of approximate volumes of the liquid 25 to a respective plurality of edible food products simultaneously. In this regard, descriptions herein of singular elements apply with equal force and effect to a plurality of the singular element and at least one of the singular element. Thus, the term “a,” “an,” “the” as used herein in connection with a singular apparatus or method step includes a plurality of the apparatus or method steps and at least one of the apparatus or method steps. Conversely, descriptions herein of plural elements apply with equal force to the singular element, or at least one of the singular element. Thus, a plural apparatus or method steps described herein includes the singular “a,” “an,” and “the,” as well as “at least one.” The dosing station 36 can be configured as an ultra-low volume liquid handling machine commercially available from Biofluidix having a place of business in Freiburg, Germany.

In one example, the support member 32 can be configured as any suitable delivery member such as a conveyor 38 or other suitable support member that is designed to support and transport the edible products to be brought into operative alignment with the dosing station 36. The conveyor 38 can be movable so as to correspondingly transport the edible product 24 from the delivery station 28 to the dosing station 36. Alternatively, the support surface 30 can be stationary, and the dosing station 36, including the applicator which can be configured as one or more dosing heads as described below, can be movable to be brought into alignment with the substrate 23. Alternatively still, both the support surface 30 and the dosing station can be movable so as to bring the dosing heads into alignment with the substrate 23. Thus, it can be said that at least one of the support surface 30 and the dosing station 36 can be movable with respect to the other of the support surface 30 and the dosing station 36 so as to bring the substrate 23 into alignment with the dosing heads of the dosing station 36.

Alternatively still, it is recognized that the system 20 can be configured for self-service whereby a user places the substrate onto the support surface 30 at the dosing station 36. Alternatively, the user can place the substrate 23 onto the support surface 30, and manually moves the substrate 23, for instance along the support surface 30, to the dosing station 36. In this example, the support surface 30 can be a stationary support surface. Further, the dosing station can be stationary.

After the active pharmaceutical ingredient has been delivered from the dosing station 36 to the substrate 23, the active-containing substrate 23 can be moved from the dosing station 36 to the post-processing station 40. The active-containing substrate 23 can be moved from the dosing station 36 to the post-processing station 40 using the support surface 30 or any suitable alternative apparatus. In this regard, the post-processing station 40 can be positioned inline with the dosing station 36 along the support surface 30 in some examples. Alternatively, the post-processing station 40 can be offline with respect to the support surface 30. Thus, the active-containing substrate can remain at the post-processing station 40 for as much time as desired until the active-containing substrate is suitable to be packaged. At that point, the active-containing substrate can be moved from the post-processing station 40 to the packaging station 42. The support surface 30 or any suitable alternative apparatus can move the active-containing substrate from the post-processing station to the packaging station 42. In this regard, the post-processing station 40 can be positioned inline with the dosing station 36 along the support surface 30, or can be offline with respect to the support surface 30.

Once the edible product 24 is aligned with the dosing station 36, the dosing station 36 is configured to deliver a predetermined approximate volume of the active pharmaceutical ingredient, such as at least one cannabinoid, to the edible product. In some examples, the active pharmaceutical ingredient can be presented as the liquid 25. Because the concentration of the active pharmaceutical ingredient in the liquid 25 is known, and the desired dose of the active pharmaceutical ingredient to be delivered to the substrate 23 is known, the approximate volume of the liquid 25 to be delivered to the substrate 23 can be determined. In some examples, electrostatic forces can be created that drive the active pharmaceutical ingredient to the substrate 23, whereby the active pharmaceutical ingredient and the substrate are oppositely charged. For instance, the substrate 23 can be provided with a negative electrical charge, and a positive charge can be applied to the liquid or powder to be delivered, thereby creating the electrostatic charge.

In other examples, it is recognized that the active pharmaceutical ingredient can be delivered to the substrate 23 as a powder. For instance, the liquid 25 containing the at least one cannabinoid can be in the initial form of a resin that can dry and crystallize. The resulting crystals can be ground into a powder having a desired dose of active pharmaceutical ingredient. Because the density of the active pharmaceutical ingredient in the powder is known, and the desired dose of the active pharmaceutical ingredient to be delivered to the substrate 23 is known, the approximate mass of the powder to be delivered to the substrate 23 by the dosing station 36 can be determined.

The dosing station 36 can include at least one applicator of the type described above, such as a plurality of applicators. Each applicator can define a dosing head 46 that is configured to dispense a respective approximate quantity of the approximate volume of the liquid 25 that is delivered from the holding tank 26. Thus, the dosing station 36 can include at least one dosing head 46 such as a plurality of dosing heads 46. The dosing station 36, and in particular the applicators and thus the dosing heads 46, is in fluid communication with the holding tank 26. Thus, the dosing heads 46 are configured to receive respective quantities of the volume of the liquid 25 delivered from the holding tank 26, and dispense the respective quantities to the edible product 24. The respective quantities dispensed by the dosing heads 46 cumulatively define the approximate volume of liquid 25 that has been received from the holding tank 26.

As will now be described, the dosing heads 46 can be configured to deliver precise quantities of the volume of the liquid 25 to the edible products 24. In some examples, the precise quantities can be microquantities applied to the edible product 24. Thus, the edible products 24 can receive a predictable dosage of the active pharmaceutical ingredient within federal regulations. Further, the dosage of the active pharmaceutical ingredient can be applied at specific locations of the edible product as desired. For instance, in certain examples, it may be desirable to deliver the active pharmaceutical ingredient such that it is substantially evenly distributed on or in the edible product 24. As a result, for instance when the edible product is a large baked good, consumption of different regions of the edible product in equal volumes will cause ingestion of substantially identical quantities of the active pharmaceutical ingredient. One non-limiting example of a large backed good can be a brownie. Further, when the edible product 24 is a bite-size food product that is the product of batch ingredients that have been mixed and/or cooked and subsequently singulated, consumption of different bite-size food products having equal volumes will cause ingestion of substantially identical quantities of the active pharmaceutical ingredient. One non-limiting example of such a bite-sized food product can be a gummy candy. In one example, the dosing heads 46 can be defined by a True Volume™ Piston Positive Displacement Pump commercially available from Creative Automation Company having a place of business in Sun Valley, Calif. In another example, the dosing heads 46 can be defined by a Pipetman MP10M device commercially available from Gilson Inc., having a place of business in Middleton, Wisc.

Referring now to FIG. 3 in particular, the dosing station 36 can include an injection reservoir 49 disposed between the holding tank 26 and the dosing heads 46. The dosing station 36 can include a first conduit 51 that extends from the holding tank 26 to the reservoir 49, and a second conduit 53 that extends from the reservoir 49 toward the dosing heads 46. Thus, the reservoir 49 can receive the volume of the liquid 25 from the holding tank 26. The dosing station 36 can further include a second conduit 53 that extends from the reservoir 49. The second conduit 53 can extend to a manifold 55. The reservoir 49 can thus deliver the volume of the liquid 25 to the manifold 55 under a pressure differential provided by a pump, and the manifold 55 can distribute the volume of liquid 25 to the dosing heads 46. In this regard, it should be appreciated that the second conduit 53 is in fluid communication with the dosing heads 46. The pump can be a positive pump that defines a positive pressure differential. The holding tank 25 can put under positive pressure so as to provide a positive force that urges the liquid 25 out of the holding tank 26 toward the dosing heads 46. Alternatively, the holding tank 25 can put under negative pressure so as to draw the liquid 25 out of the holding tank 26 toward the dosing heads 46. In other examples, the system 20 can include a plurality of pumps that are each configured to provide a respective pressure differential to a respective one or more of the dosing heads 46.

The pumps can, for instance, define respective pistons that are movable in corresponding cylinders to eject a predetermined precise volume of the liquid 25. In this regard, the stroke length of the piston that delivers the liquid 25 to a first at least one dosing head 46 can be different than the stroke length of the piston that delivers liquid 25 to a second at least one dosing head 46. Alternatively, the pumps can include an elastic micropipe with an inner diameter that is partially squeezed by a piezo stack actuator so as to drive the liquid 25 out of the dosing head 46.

In some examples, different dosing heads 46 can be configured to deliver different quantities of the respective volume of liquid 25 to the edible product 24 (see FIG. 2). Further, the liquid 25 delivered by the first at least one dosing head 46 can include a different active pharmaceutical agent than the liquid 25 delivered by the second at least one dosing head 46. Further still, the system 20 can be configured to deliver any number of API-containing liquids 25 each containing a different pharmaceutical agent to a respective at least one dosing head 46. Accordingly, the dosing heads 46 can combine to deliver active pharmaceutical agents from different liquid extracts in different quantities onto a common substrate 23. Alternatively or additionally, the different liquids can have different concentrations of their respective active pharmaceutical agent. The system 20 can therefore include any number of holding tanks 26 as desired, each tank containing a different liquid extract that contains a different at least one pharmaceutical active ingredient. The different liquid extracts can be delivered to different respective ones of the dosing heads 46. Thus, different dosing heads can be configured to deliver different cannabinoids to the substrate.

As one example, a first group of dosing heads 46 can be configured to deliver a dose of a first active pharmaceutical agent, and a second group of dosing heads 46 can be configured to deliver a dose of a second active pharmaceutical agent, wherein the second active pharmaceutical agent is different than the first active pharmaceutical agent. For instance, the first active pharmaceutical agent can be THC, and the second active pharmaceutical agent can be CBD. Further, the first active pharmaceutical agent can be delivered in a different predetermined approximate dose than the second active pharmaceutical agent. Further still, the tank containing the first active pharmaceutical agent can be maintained at a different temperature than the second tank. Thus, the viscosity of each of the respective API-containing liquids 25 can be individually controlled. Additionally, the temperature of one or more up to all of the respective conduits 51 and 53 and at the respective dosing heads 46 can be different so as to individually control the viscosity of each liquid extract as it travels from the respective tank to the respective one or more dosing heads 46.

The system 20 can include any suitable feedback mechanism to provide an indication that the at least one dosing head 46 has delivered the at least one active pharmaceutical ingredient to the substrate 23. The feedback mechanism can be a closed feedback loop in some examples. For instance, a pressure sensor can be placed in the conduit 53 so as to measure the backpressure in the conduit 53. A drop in the backpressure, for instance, can indicate that the respective at least one dosing head 46 has delivered the respective at least one active pharmaceutical ingredient to the substrate 23. Alternatively, the system 20 can include a load cell that determines, by sensing weight, that the substrate 23 is in alignment with the dosing head 46. Alternatively still, the system 20 can include a visual recognition system that includes a visual sensor to visually identify that the substrate 23 is in alignment with the dosing head 46. It is therefore appreciated in some examples, that the substrates 23 can be positioned at any location on the support surface that need not be a predetermined location of the support surface.

Further, the system 20 can include a camera that is designed to measure a quantification of the microdroplets delivered from the dosing heads 46. For instance, the camera can measure a cross-sectional dimension of the microdroplets as they travel from the dosing heads 46 to the substrate 23. It is recognized microdroplets can be elongated as they travel out of the dosing heads. However, the surface tension of the microdroplets can cause the microdroplets to become more spherical as they travel from the dosing heads 46 to the substrate. Thus, in one example, the cross-sectional dimension can be a maximum cross-sectional dimension that approximates the diameter of a sphere, such that an approximation of the volume of the microdroplets can be calculated if desired. However, the cross-sectional dimension can be any suitable alternative cross sectional dimension that has a relationship to the volume of the microdroplet. The cross-sectional dimensions or calculated approximations of the volumes of the microdroplets can be compared to each other so as to ensure consistency of the volume of microdroplets being delivered to the substrates 23, or to verify a desired variation in the volumes of microdroplets. The cross-sectional dimensions or calculated approximations can then be integrated into the feedback look to ensure proper operation of the system 20. In one example, the camera can be a SmartDrop System commercially available from Biofluidix having a place of business in Freiburg, Germany.

As described above, the system 20 can be configured to deliver heat to the liquid 25 either in one or more of the conduits and/or in the dosing head 46 prior to or during dispensing of the API containing liquid to the substrate 23. The heat can be sufficient to decrease the viscosity of the API-containing liquid 25. In some examples, for instance when the API-containing liquid 25 includes a solvent, the step of delivering heat to the liquid 25 can cause the solvent to evaporate, such that pure API having a sufficiently low viscosity is dispensed from the dosing heads 46. Thus, in one example, the API-containing liquid 25 can include the API and solvent, and can travel from the holding tank 26 to the dosing head 46. The API-containing liquid 25 can be heated between the holding tank 26 and the dosing head 46 to decrease the viscosity of the liquid 25 and, in some instances, evaporate some or all of the solvent. Alternatively or additionally, the API-containing liquid 25 can be heated at the dosing head 46 so as to decrease the viscosity of the liquid 25 and, in some instances, evaporate some or all of the solvent. In one example, the system 20 can include at least one heater that delivers heat to one or more up to all of the first conduit 51, the second conduit 53, the injection reservoir 49, the manifold 55, and the dosing head 46, so as to decrease the viscosity of the API-containing liquid and, in some instances, evaporate the solvent. In one example, the liquid 25 can be maintained at a temperature in a range from approximately 100 degrees F. to approximately 200 degrees F., such as from approximately 140 degrees F. to approximately 200 degrees F., and in one example from approximately 150 degrees F. to approximately 180 degrees F. Alternatively, in some examples such as when the liquid 25 is a solution, the liquid 25 can be maintained at room temperature.

While the dosing heads 46 can be configured to deliver to the substrate 23 the liquid 25 that contains at least one active pharmaceutical ingredient in one example, the dosing heads 46 can alternatively be configured to deliver the at least one active pharmaceutical ingredient to the substrate 23 in the form of a solid or powder in the manner described herein with respect to the liquid 25. Thus, examples above of applying the active pharmaceutical ingredient in the form of a liquid can apply with equal force and effect to a powder including the at least one active pharmaceutical ingredient, unless otherwise indicated. Each dosing head 46 can be configured to deliver microdroplets of the API as described above. Thus, it should be appreciated the powder can be delivered to the substrate 23 as a microquantity. The powder can be stored in the holding tank 26, and can be directed through the first conduit 51 and the second conduit 53 to the dosing head 46, either directly or through the manifold 55. Thus, it can be said that a quantity of API containing material can be applied to the substrate 23. The API containing material can be in the form of a powder or a liquid. Thus, the API containing material can include a desired concentration of active pharmaceutical ingredient as described above. In other examples, the API containing material can include only the active pharmaceutical ingredient.

Further still, while each of the dosing head 46 can be configured to dispense the API-containing liquid 25 that has been received from the holding tank 26, it is recognized that the API-containing liquid 25 can be delivered using other methods. For instance, the system 20 can include a first holding tank that contains the API in liquid or solid form, and a second holding tank that contains a solvent. The API and solvent can mix at the dosing station 36. For instance, the API and solvent can mix at the dosing head 46. In one example, the dosing head can include a first chamber that receives the API, and a second chamber that receives the solvent. The API and solvent can mix in the dosing head 46 to produce a solution having the predetermined concentration of API. The solution produced in the dosing head 46 can then be dispensed as one or more microdroplets in the manner described herein. In some examples, the concentration can be varied inside the dosing head 46. That is, the respective proportions of API and solvent that are mixed in the dosing head 46 can be varied. Further, the API or the solution can be mixed with at least one other ingestible modifier that is configured to modify at least one of flavor, one or more mechanical properties, or one or more aesthetics of the cannabis or hemp material. The mixing can occur in the dosing head 46 or at any other location as desired. For instance, the at least one other edible product can be mixed in the liquid 25 in the holding tank 26 in some examples.

Referring again to FIGS. 2A-3B, in one example, the dosing heads 46 can be arranged in an array 48 that includes at least one row 50 of dosing heads 46. The dosing heads 46 of each row 50 can be substantially equidistantly spaced along the respective row 50. Alternatively, the dosing heads 46 can be variably spaced along the respective row 50. The array 48 can further include a plurality of columns 52 that spaces the rows 50 from each other. The dosing heads 46 can be equidistantly spaced along the respective columns 52. Alternatively, the dosing heads 46 can be variably spaced along the respective columns 52. In one example, all of the dosing heads 46 can be configured to deliver the same at least one active pharmaceutical ingredient. Alternatively, as described above, different groups of the dosing heads 46 can be configured to deliver respective different active pharmaceutical ingredients. Each group can include at least one dosing head 46 up to a plurality of the dosing heads 46. Each group can be defined by a respective one or more of the rows 50. Alternatively, each group can be defined by a respective one of the columns.

Referring now to FIGS. 2A-4, the dosing heads 46 can be aligned with different respective locations of a dosing zone 54 the edible product 24. Accordingly, the dosing heads 46 can be positioned to deliver their respective quantities of the volume of liquid 25 to the different respective locations of the dosing zone 54. Further, the system 20 can be configured to deactivate select dosing heads 46 that are out of alignment with the dosing zone 54 and thus do not receive respective portions of the volume of liquid 25, and activate select dosing heads 46 that are aligned with the dosing zone 54 and thus receive respective portions of the volume of liquid 25. In some examples, the system 20 can include a sensor that identifies the dosing zone 54 of the edible product 24. The sensor can be a camera, a weight sensor that measures the weight of the substrate 23 on the support surface and determines the dosing zone based on the weight and/or size, or any suitable alternative sensor. The dosing zone 54 can be at least partially defined by an outer perimeter 56 of the edible product 24. For instance, the dosing zone 54 can be defined in its entirety by the outer perimeter 56 of the edible product 24. Thus, an entire outer surface of the edible product 24 can define the dosing zone 54. In some examples, the dosing zone 54 can be disposed inside the outer perimeter 56 in its entirety. For instance, the dosing zone 54 can be greater than half, for instance greater than 75%, of a footprint defined by the outer perimeter. Either way, it can be said that the dosing zone 54 can be a substantially predetermined location with respect to the outer perimeter 56 of the edible product 24. Thus, the dosing zone 54 can be consistent among a plurality of differently sized edible products 24, such as cookies or brownies that can have similar but non-identical sizes and shapes.

The dosing heads 46 can be spaced from each other as desired so as to deliver a desired distribution of the active pharmaceutical ingredient to the edible product 24 in the dosing zone 54. Alternatively, one or more dosing heads 46 can be movable so as to deliver the active pharmaceutical ingredient to multiple locations of the edible product 24. In one example, the dosing heads 46 are configured to deliver a substantially even distribution of the volume of liquid 25 to the edible product 24 in the dosing zone 54. For instance, the respective quantity of the volume of suspension dispensed by each of the dosing heads 46 or each plurality of dosing heads can be substantially equal to the respective quantity of suspension dispensed by the other dosing heads 46 or other pluralities of dosing heads 46.

In another example, the system 20 can divide the dosing zone 54 into a plurality of subzones. Each subzone can be configured to receive a different at least one active pharmaceutical ingredient. Thus, a first group of at least one dosing head 46 can deliver a first at least one active pharmaceutical ingredient to a first one of the subzones, and a second group of at least one dosing head 46 can deliver a second at least one active pharmaceutical ingredient that is different than the first at least one active pharmaceutical ingredient to a second one of the subzones. Alternatively or additionally, the first group of at least one dosing head 46 can be configured to deliver a first dose of the first at least one active pharmaceutical ingredient, and the second group of at least one dosing head 46 can be configured to deliver a second dose of the second at least one active pharmaceutical ingredient that is different than the first dose. In still another example, the first and second groups of at least one dosing head 46 can be configured to deliver the same at least one active pharmaceutical ingredient, but in different doses. The active pharmaceutical ingredient can be substantially evenly distributed in each of the subzones.

In some examples, at least one dosing head 46 such as a plurality of dosing heads 46 can be movable along the substrate 23 so as to deliver the respective at least one active pharmaceutical ingredient at different locations of the edible product 24. Further, the dosing heads 46 can be configured to deliver different active pharmaceutical ingredients to the substrate 23. For instance, the dosing heads 46 can be configured to deliver different combinations of liquids and/or powders. In one example, the dosing heads 46 can deliver to the substrate 23 a first liquid or powder that contains a first active pharmaceutical ingredient. Next, the dosing heads 46 can deliver to the substrate 23 a second active pharmaceutical ingredient that is different than the first active pharmaceutical ingredient. Next, the dosing heads 46 can deliver to the substrate 23 a third active pharmaceutical ingredient that is different from each of the first and second active pharmaceutical ingredients, and so forth until all desired active pharmaceutical ingredients have been delivered to the substrate 23.

When the dosing heads 46 are arranged in groups of dosing heads 46 that each deliver a respective different at least one active pharmaceutical ingredient, the different active pharmaceutical ingredients can be delivered to respective different locations of the substrate 20. For instance, the dosing heads 46 can remain stationary with respect to the substrate 23 as the active pharmaceutical ingredient is delivered to the substrate 23. Alternatively, the dosing heads 46 can be movable along the substrate 23, such that the combination of active pharmaceutical ingredients as delivered by different groups of at least one dosing head 46 can be delivered to the same respective location of the substrate 20. The heads 46 can be movable such that the dosing heads 46 can deliver the respective active pharmaceutical ingredient to different respective locations of the substrate 23 than the other dosing heads. The active pharmaceutical ingredients in the different respective locations can be substantially evenly distributed in at least one direction along to the substrate 23. For instance, the active pharmaceutical ingredient in the different locations can be substantially evenly distributed in two perpendicular directions along the substrate 23.

The substrate 23 includes an external surface that defines an inner surface 60 that faces the support surface 30, and an outer surface 58 that is opposite the inner surface 60. The dosing heads can deliver the active pharmaceutical ingredient to the outer surface 58 of the substrate 23. The edible product 24 defines a thickness that extends from the inner surface 60 to the outer surface 58. The delivered volume of active pharmaceutical ingredient can substantially remain on the outer surface 58. Delivering the volume of liquid to the outer surface 58 can expose the liquid to oral receptors, thereby increasing speed of ingestion of the active pharmaceutical ingredient. Alternatively or additionally, the delivered volume of liquid 25 can permeate through the outer surface 58 so as to impregnate at least a volume of a thickness of the edible product that extends from the outer surface 58 to the opposed inner surface 60. Alternatively still, the active pharmaceutical ingredient can be injected into the substrate 23 between the inner surface 60 and the outer surface 58. For example, at least 20% of the active pharmaceutical ingredient can be disposed in a middle 75% of the thickness. The middle 75% of the thickness can be equidistantly spaced from each of the inner surface 60 and the outer surface 58. For instance, at least 20% of the active pharmaceutical ingredient can be disposed in a middle 50% of the thickness. The middle 50% of the thickness can be equidistantly spaced from each of the inner surface 60 and the outer surface 58. In some examples, the distribution along the outer surface of the substrate 23 can be different than the distribution along the thickness of the substrate 23 from the outer surface to the inner surface.

In one example, the dosing heads 46 can be configured to deliver the respective quantities of the active pharmaceutical ingredient to the respective locations of the outer surface 58 of the edible product 24 in the form of microdroplets 62. The microdroplets 62 can have any suitable size and shape as desired. In one example, the microdroplets 62 can including microquantities of the active pharmaceutical ingredient. For instance, the microdroplets 62 can define a maximum cross-sectional dimension along a horizontal direction that is in a range from approximately 5 millionths of an inch, for instance when printed, up to approximately 100 thousandths of an inch. For instance, the range can be from approximately 5 thousandths of an inch to approximately 50 thousandths of an inch. In one example, the maximum cross-sectional dimension along the select direction can be in a range from approximately 20 thousandths of an inch to approximately 40 thousandths of an inch. The dosing heads 46 are spaced from the edible products 24 along a direction of travel of the active pharmaceutical ingredient from the dosing heads 46 to the edible products 24. Thus, the active pharmaceutical ingredient is delivered to the substrate along the direction of travel. The select direction can be substantially perpendicular to the direction of travel. In one example, the dosing heads 46 are spaced above the edible products 24 along a vertical direction. Thus, the select direction can be a substantially horizontal direction. For instance, the dosing heads 46 can be spaced from the edible products 24 any suitable distance when delivering the active pharmaceutical ingredient to the edible products 24, such as from approximately 2 mm to approximately 25 mm. As shown at FIG. 3B, at least some of the microdroplets 62 up to all of the microdroplets 62 can be substantially spherical shaped. Alternatively or additionally, as shown at FIG. 3C, at least some of the microdroplets 62 up to all of the microdroplets 62 can be elongated, for instance substantially teardrop shaped or alternatively shaped as desired.

In one example, the microdroplets 62 are delivered from the dosing heads 46 to the respective locations of the edible product 24 under any suitable force, such as gravitational forces, electrostatic forces, or the like. In another example, the microdroplets 62 are delivered from the dosing heads 46 to the respective locations of the edible product under positive pressure. In this regard, the dosing station 36 can control whether the microdroplets 62 remain on the outer surface 58 of the edible product 24, and whether the microdroplets 62 permeate through the outer surface 58 into the thickness of the edible product 24 in the manner described above. In still other examples, one or more of the dosing heads 46 can be coupled to a respective needle that can be driven into the edible product 24 so as to deliver the respective quantity of the volume of liquid 25 into the edible product 24 at a location between the outer surface 58 and the inner surface 60. In some instances, the needle can be heated at a temperature suitable to soften or melt locations of the substrate contacted by the needle, in order to assist in the injection of the needle into the substrate. The heated needle can also maintain a desired viscosity of the at least one active pharmaceutical ingredient as the active pharmaceutical ingredient is being delivered through the needle and into the substrate. Whether the active pharmaceutical ingredient is delivered to the edible product 24 as microdroplets or as an injection, the active pharmaceutical ingredient can be delivered to the edible product in microquantities.

As described above, the system 20 can include the post-processing station 40 that is configured to process the edible product 24 after the liquid 25 has been delivered to the edible product 24. The post-processing station 40 can be configured to dry the solvent, for instance, when the API is delivered as a solution. In this regard, the post-processing station 40 can include any suitable drying member, such as at least one drying head 70 or a plurality of drying heads 70 that are configured to deliver a drying agent to the respective locations of the edible product 24 so as to dry the liquid 25. It is appreciated that when the liquid 25 dries, the solvent of the delivered volume of liquid 25 that carries the active pharmaceutical ingredient also dries and can evaporate, leaving the active pharmaceutical ingredient on the substrate 23. In this regard, the drying heads 70 can be arranged in an array that has an equal number of rows and columns as the array of dosing heads 46. Further, the relative position of the drying heads 70 with respect to the other drying heads 70 can be the same as the relative position of the dosing heads 46 with respect to the other dosing heads 46. Thus, the drying heads 70 can be aligned with the active pharmaceutical ingredients that were delivered to the edible product 24 by the dosing heads 46.

The drying agent can be configured as any suitable light, including ultraviolet, laser, infrared, or the like. Alternatively, the drying agent can be a forced gas that is delivered to the outer surface of the edible product 24. The forced gas can be air, nitrogen, or any suitable alternative gas such as an inert gas. The forced gas can be heated, and can have a temperature that is in a range for instance from about 100 degrees F. to about 250 degrees F. Alternatively, the forced gas can be substantially unheated, and thus at ambient temperature. Alternatively still, the forced gas can be cooled, and thus at a temperature below ambient temperature. In this regard, the cooled forced gas can cause a cannabinoid to freeze on the surface of the substrate, or to delay evaporation of the solvent so as to allow the cannabis-containing solution to further impregnate the thickness of the substrate 23. Alternatively, the post-processing station 40 can expose the dosed substrate to ambient air or a controlled environment so as to dry the volume of liquid 25. It is recognized that the drying agent applied to the API can increase the viscosity of the API. The post-processing station can further cause the API to further adhere to the substrate 23. For instance, increasing the viscosity can cause the API to further adhere to the substrate 23. Further, applying forced air to the substrate 23 can cause the API to disperse along the substrate as the API travels along the outer surface of the substrate 23, thereby facilitating absorption of the API into the substrate 23. For instance, it is recognized that the API can become saturated in the portion of the substrate 23 that underlies the delivered microdroplets. Causing the API to move along the outer surface of the substrate 23 then allows the API to absorb into the substrate 23 at locations of the substrate 23 that are not saturated with the API. The post-processing station 40 can further cause the API to solidify on or in the substrate 23. In some examples, the API can crystallize on or in the substrate 23. Alternatively, the API can remain as an oil on or in the substrate 23. If the at least one cannabinoid is applied as a powder, the post-processing step can apply heat to the powder, thereby causing the at least one cannabinoid to liquify on the substrate 23. Subsequent cooling of the liquified powder can cause the liquid to solidify or crystallize or otherwise adhere on or in the substrate 23.

It is appreciated that energy can be applied to the substrate 23 to improve diffusion or absorption of the API into the substrate 23. For instance, when heat is applied to the surface of certain substrates 23, and in particular certain edible products, such as chocolate, baked goods, gummy candy, lollipop, and the like, the temperature of the surface of the edible product be raised to a level whereby the edible product melts, sweats, or otherwise assumes a form that is configured to encapsulate the API. The temperature can be increased, for instance, by directing at least one of heated forced air and a light to the surface.

Once the substrate 23 has been post-processed, the active-containing edible product 24 can be transferred from the post-processing station 40 to the packing station 42. At the packaging station 42, the dried edible products 24 can be individually wrapped in any suitable package 73. Alternatively or additionally, a plurality of the active-containing edible products 24 can be wrapped in a common package. The active-containing edible product 24 can include a cooked edible product, and a dose of an active pharmaceutical ingredient carried by the cooked edible product in the dosing zone of the cooked edible product. The dose of the active pharmaceutical ingredient can be substantially evenly distributed in the dosing zone. Because the edible product was fully cooked prior to adding the active pharmaceutical ingredient, the active pharmaceutical ingredient need not be cooked after the active pharmaceutical ingredient was added.

In some examples, the edible product 24 can be configured as a plurality of nuts 37 (FIG. 4A) and/or fruits 39 (FIG. 4B) and/or a mixture of dried fruits and nuts and potentially other additional food product It is appreciated that the API is not visible in FIGS. 5A-5E due to the nature of the figures. The active pharmaceutical ingredient can be applied to the nuts and fruits in any suitable manner as disclosed herein. In some examples, the nuts have been cooked, such as roasted. In other examples, the nuts can be raw. In some instances, the nuts or fruit can be prepared with salt, sugar, honey, or any suitable alternative ingredient. Thus, the nuts can be candied. In some examples, the fruit can be a raw fruit. In other examples, the fruit can be dried. In still other examples, the fruit can be candied. It is understood that fruits and nuts can have relatively low surface areas and volumes. Thus, variations in the dosage of active pharmaceutical ingredient applied to fruits and nuts can have a greater impact on the ratio of active pharmaceutical ingredient per volume of edible product when compared to edible products having larger surface areas and volumes.

Therefore, it can be particularly advantageous to precisely control the dosage of active pharmaceutical ingredient added to fruits and nuts. The active pharmaceutical ingredients can be applied to the fruits and nuts as microquantities in the manner described above, which allows for the precise control of the dosage of active pharmaceutical ingredients applied to the fruits and nuts. Depending on the size of the fruit or nut, it is recognized that microdroplets having respective volumes ranging from approximately 5 nanoliters to approximately 20 microliters can be delivered to an individual fruit or nut. Thus, each fruit or nut can include a quantity or dosage of API in the range from approximately 2.5 micrograms to approximately 20 milligrams. Therefore, each microdroplet can contain a microquantity of API in a range from approximately 0.5 micrograms to approximately 1 milligram. It is recognized, of course, that the dosage of API per dried fruit or nut can vary as desired. For instance, other quantities of microdroplets can be delivered to fruits and nuts, for instance depending on the size of the fruit and nut, the size of the microdroplet, and the concentration of API in the microdroplet. The microquantity of API in a microdroplet can allow the dosage of API delivered to a substrate to be precisely controlled as described above. Further, the dosage of API per dried fruit or nut can be precisely controlled, as can a plurality of dried fruits and/or nuts that amount to a serving. For larger edible products, such as baked goods 41 (see FIG. 5C), the microdroplets can be applied in the range of approximately 5 nanoliters to approximately 20 microliters over the entire surface and greatly increase the total API delivered to the substrate to 100 milligrams or more.

It should be appreciated that several advantages can be achieved using the system 20. In one example, the substrate 23 can include multiple active pharmaceutical ingredients, which can eliminate a conventional need to consume multiple medications each having a single active pharmaceutical ingredient. Further, microquantities of the active pharmaceutical ingredients can be applied to the substrate. Thus, the dosage of the at least one active pharmaceutical ingredient carried by the substrate can be better controlled with respect to conventional application processes. Further, the at least one active pharmaceutical ingredients can be distributed substantially evenly along the dosing zone. Further still, individual dosing of the active pharmaceutical ingredients on the substrate can allow for the use of locally produced active pharmaceutical ingredients that are applied after the substrate has been prepared, thereby avoiding the need to transport the applied active pharmaceutical ingredients across jurisdictional boundaries, which can be illegal in some jurisdictions or carry tax penalties. Further, dosing the substrate after the substrate has crossed the jurisdictional boundary can reduce or eliminate degradation of the active pharmaceutical ingredient during transportation across the jurisdictional boundary, which can sometimes involve long distances of transportation. In some examples, a dye can be used with the active pharmaceutical ingredient if desired, so as to confirm that the active pharmaceutical ingredient has been delivered to the substrate.

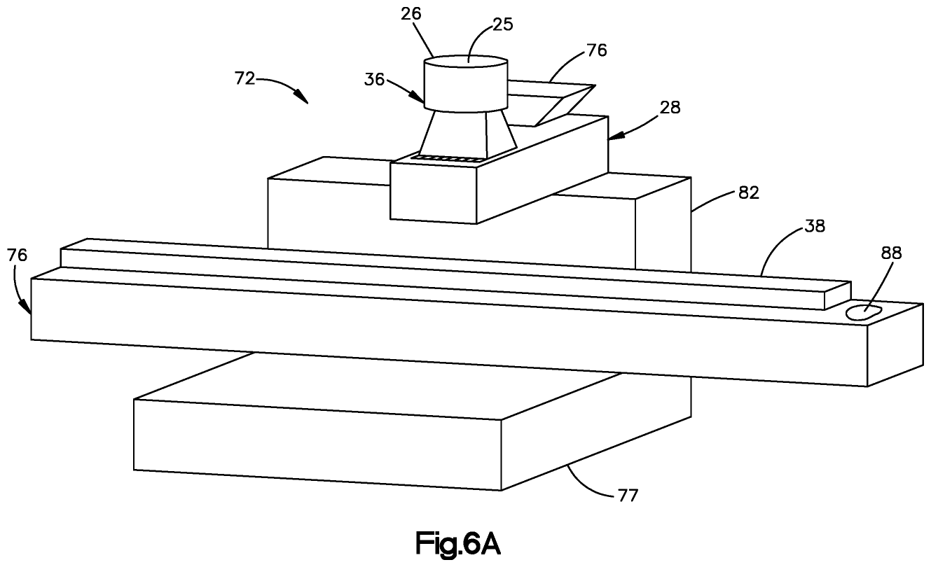

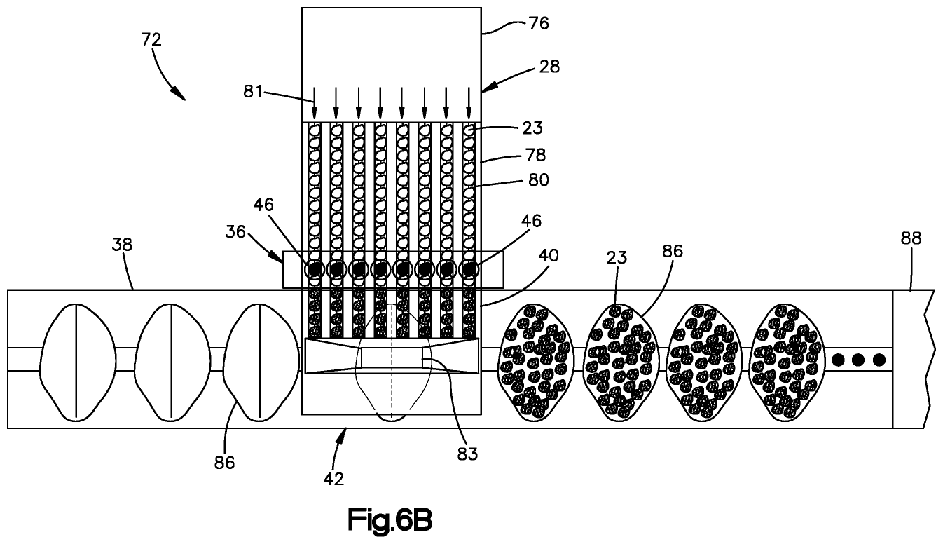

Referring now to FIGS. 6A-6B, it is recognized that in some examples the system 20 described above can be configured as a single unitary stand-alone dosing machine 72. The dosing machine 74 can include the conveyor 38, the holding tank 26, the delivery station 28, the dosing station 36, the post-processing station 40, and the packaging station 42. The dosing machine 74 can further include a support structure 76 that supports the conveyor 38, the holding tank 26, the delivery station 28, the dosing station 36, the post-processing station 40, and the packaging station 42. Thus, the conveyor 38, the holding tank 26, the delivery station 28, the dosing station 36, the post-processing station 40, and the packaging station 42 can be said to be integrated into the single stand-alone dosing machine, and supported by the common support structure 76. The dosing machine 72 can further include the camera that measures the maximum cross-sectional dimension of the microdroplets in the manner described above. Further, the system 20, and dosing machine 74, can include a cleaner that is configured to remove loose particulates from the substrate 23 prior to delivering the API to the substrate 23. For instance, in the case of salted nuts, loose salt can be removed from the nuts, while salt having strong adhesion to the nuts remain. In one example, the cleaner can be configured to deliver forced air to the substrate to remove loose debris from the substrate. Thus, when the API is delivered to the substrate, the API can have strong adhesion to the substrates 23. In some examples, the forced air can be heated so as to increase the temperature of the substrate so as to improve absorption or diffusion of the API into the substrate in the manner described herein.

The delivery station 28 can include a hopper 76 or other containment member that contains one or more of the substrates 23. The delivery station 28 can further include a delivery member 78 that is configured to receive the substrates from the hopper 76, and deliver the substrates 23 from the hopper 76 to the dosing station 36. For instance, the delivery member 78 can transport the substrates 23 from the hopper 76 to the dosing station 36, and further to a third location in alignment with a delivery location whereby the dosed substrates are delivered to the conveyor 38. The delivery member 78 can include define any suitable material that can have at least one elongated groove 80 or other suitable structure that directs the substrates 23 along a respective path 81 from the hopper 76 to the dosing station 36. For instance, the delivery member 78 can include a plurality of grooves 80 that define a plurality of paths 81 from the hopper 76 to a plurality of respective dosing stations 36. Alternatively, a plurality of delivery members 78 can define respective grooves 80 that extend along respective paths from the hopper 76 to the respective dosing stations 36.

In one example, the delivery member 78 can be downwardly sloped along a direction from a first location in alignment with the hopper 76 to a second location in alignment with the dosing station 36, and further to a third location in alignment with a delivery location whereby the dosed substrates are delivered to the conveyor 38. Further, the delivery member 78 can be configured to vibrate, shake, or otherwise cause the substrates 23 to travel along the delivery member 78 from the first location to the second location, and from the second location to the third location. Alternatively, the delivery member 78 can be configured as a conveyor so as to move the substrates 23 from the first location to the second location, and from the second location to the third location. Alternatively still, as noted above, a user can manually move the substrates 23 along the delivery member 78 or otherwise move the substrates to a position in alignment with the dosing station 36.

During operation, the substrates 23 loaded into the hopper 76. The substrates 23 are then delivered from the hopper 76 to the first location of the delivery member 78. The delivery can occur under gravitational forces or any suitable alternative structure and method. In particular, the substrates 23 can be delivered to the delivery member 78 such that they are arranged along their respective paths 81. The substrates 23 can be individualized and arranged in single file on their respective delivery members 78, and thus along their respective paths 81. Alternatively, groups of the substrates 23 can be arranged on one or more of the delivery members 78. The substrates 23 traveling along the respective paths 81 can define the same type of substrate, such as fruits or nuts, or a baked good or the like. Alternatively, the different types of substrate 23 can travel along the respective paths 81. For instance, the substrates 23 traveling along one path can include dried fruits. The substrates 23 traveling along another path or the same path can include nuts, either raw or roasted. The substrates 23 traveling along still another path can include a baked good.

The substrates 23 travel along the delivery member 78 to the second location, whereby the substrates 23 are aligned with the respective a least one dosing station 36. The dosing machine 72 can include a plurality of dosing stations 36, whereby each of the dosing stations 36 is aligned with a respective one of the delivery members 78. As described above, each of the delivery members 78 causes extends along a respective path 81. Thus, each of the dosing stations 36 is aligned with one of the respective paths 81, and is configured to deliver API to the substrates 23 that travel along the respective paths 81. In one example, the substrates 23 can be arranged on the delivery member 78 such that one or more dosing heads 46 of the dosing stations 36 aligned with the respective paths are configured to deliver API to only a single substrate 23 at a time as the substrates 23 travel along the respective paths 81. In particular, the dosing heads 46 can be configured to deliver microdroplets to each individual substrate 23 in the manner described herein. Because the API is delivered in microdroplets, a precise predetermined dosage of API is delivered to each of the substrates 23.

In one example, the same API-containing liquid can be delivered to a plurality of the substrates 23. Alternatively, API-containing liquids having different API characteristics can be delivered to different substrates 23. The different substrates 23 can define respective pluralities of substrates. The pluralities of substrates can travel along different respective paths 81 to different dosing stations 36 that are operably aligned with the respective paths 81. The dosing stations 36 can deliver respective APIs to the aligned substrates 23, either individually or as a group of substrates 23, where the respective APIs have at least one API characteristic that is different than the other. Alternatively, the pluralities of substrates 23 can travel along the same path 81 to the same dosing station 36. Alternatively still, the pluralities of substrates 23 can travel along different paths 81 to the same dosing station 36. The same dosing station can deliver a first API-containing liquid to a first at least one substrate 23, such as a first plurality of substrates 23. The same dosing station can deliver a second API-containing liquid to a second at least one substrate 23, such as a second plurality of substrates 23. The first and second APIs can have at least one API characteristic that is different from each other. The different API characteristic can include at least one of 1) a concentration of the API, 2) a volume of API delivered to the substrates during the delivering step, which can include at least one of a different number of microdroplets and microdroplets having different volumes, 3) a composition of the API, 4) a modifier mixed with the API, the modifier configured to modify at least one of a flavor, a mechanical property, and an aesthetics of the delivered API, and 5) a location of at least one dosing zone of the substrate 23 that defines a location of the substrate 23 where the API-containing liquid is to be deposited. The mechanical property can include viscosity of the API-containing liquid in some examples. The mechanical property can further include the surface tension of the API-containing liquid that is delivered from the dosing station. It is further appreciated that the different API characteristic can include a different predetermined dosage that is delivered to the different substrates 23. In one example, the dosage can be predetermined to correspond to a dosing regimen over a period of time. Thus, groups of one or more substrates can have dosages that differ and are designed to be consumed at predetermined times over the course of the dosing regimen. For instance, the dosage can decrease over the period of time defined by the dosing regimen. Alternatively, each substrate 23 can receive API-containing liquid 25 having the same API characteristics. Further, different groups of substrates can receive API-containing liquid 25 with at least one different API characteristic, wherein all substrates among each group receive the same API-containing liquid 25.

Alternatively, it is envisioned that a predetermined quantity of substrates 23, or a plurality of substrates 23, such as dried fruits and/or nuts, can be grouped together on the delivery member 78 along the respective path 81. Thus, the dosing stations 36 can be aligned with the plurality of substrates 23 of the group. The dosing heads 46 can thus deliver a predetermined or target quantity of API-containing microdroplets to the group as a whole as opposed to each individua dried fruit or nut. Because the API is delivered in microdroplets, a precise predetermined dosage of API is delivered to the group of substrates 23. The group of substrates 23 can be intended to be ingested in a single serving. Thus, the precise predetermined dosage is ingested when the substrates 23 of the group are ingested. It is recognized that the predetermined quantity of substrates 23 dosed in a group are not limited to fruits and nuts, but to any type of edible product 24 that is designed to be consumed in quantities, such as chips, popcorn, pretzels, candies such as gummy candy 45 (FIG. 4D), and the like. It should therefore be appreciated that the dosing station can be configured to deliver an API to at least one substrate 23 at a time, which can include the single substrate 23 or the group of substrates 23.

Each dosing station 36 can include at least one dosing head 46, such as an array of dosing heads 46, and one or more holding tanks 26 that can contain a respective API-containing liquid 25 described above. It is recognized that a plurality of dosing stations 36 can receive the API-containing liquid 25 from a common one of the holding tanks. Alternatively, the dosing stations 36 can receive API-containing liquid 25 from a different holding tank 26. The API-containing liquids 25 in the different holding tanks 26 can have different APIs from each other or the same API. Thus, the API can be a cannabinoid or any suitable alternative active pharmaceutical ingredient. Each at least one dosing head 46 of the dosing station 36 can be operatively aligned with a respective one of the paths 81 so as to deliver the active pharmaceutical ingredient to the at least one substrate 23 traveling along the respective one of the paths 81. Accordingly, as the at least one substrate 23 travels to the second location along the respective path, the dosing station 36 delivers a predetermined quantity of the API-containing liquid 25 from the at least one dosing head 46 to the respective aligned at least one substrate 23. The dosing head 46 discontinues delivery of the API-containing liquid 25 when the at least one substrate 23 has received the predetermined quantity of the liquid 25. The dosing heads 46 of the array of dosing heads 46 can combine to deliver the predetermined quantity of the liquid 25 to respective different locations of the at least one substrate 23. That is, the locations of the at least one substrate 23 can be aligned with different dosing heads 46 of the array of dosing heads 46 that are aligned with the respective path 81.

After each at least one substrate 23 has received the predetermined quantity of liquid 25, the substrates 23 move along the delivery member 78 past the second location. The dosing stations 36 resume delivery of the liquid 25 when another at least one substrate 23 has traveled to the second location to a location in alignment with the at least one dosing head 46. In this regard, the at least one substrate 23 arranged sequentially along each respective paths 81 receive the predetermined quantity of liquid 25 from the aligned one of the dosing stations 36. The predetermined quantity can be equal to each other or different than each other as desired, depending on the at least one substrate 23 and the desired dosage of the active pharmaceutical ingredient that is to be delivered to the at least one substrate 23. The dosing machine 72 can include a processor programmed with the dosage quantities that are to be applied to the substrates 23 traveling along the at least one delivery member 78. The processor can control the operation of the delivery member 78 and each of the stations of the machine 72 described above. For instance, the dosing station 36 can include any suitable apparatus or sensor as described above to identify when one of the substrates 23 has traveled into alignment with the dosing head 46, and when no substrates 23 are aligned with the dosing head 46 that require delivery of liquid 25, and communicate the alignment information to the processor. The processor then controls operation of the dosing heads 48.

Once the substrates 23 have been dosed with the active pharmaceutical ingredient-containing liquid 25, the substrates 23 travel along the delivery member 78 to the third location whereby they are delivered to the conveyor 38. In this regard, the delivery member 78 can be disposed in a spatial relationship with respect to the conveyor, such that the substrates 23 can travel from the delivery member 78 to the conveyor 38. In one example, the delivery member 78 is supported by a delivery support member 82 of the dosing machine 72. Thus, the support structure 76 can include a base 77 that supports the conveyor 38 and packaging station 42, and the delivery support member 82 that supports the delivery member 78 in addition to the dosing station 36, at least one holding tank 26, the at least one hopper 76, and the post-processing station 40. The substrates 23 can be dried as they travel from the second location to the third location. Thus, the dosing machine 72 can include a post-processing station 40 between the second location and the third location. The post-processing station can be configured as described above. Thus, once the substrate 23 is processed during the processing step, the API can adhere to the substrate 23.

The delivery support member 82 can support the delivery member 78 at a location above the conveyor 38, such that the dosed substrates 23 can travel from the support structure 78 down toward the conveyor 38. In one example, the dosed substrates 23 can travel under gravitational forces from the support structure 78 toward the conveyor 38. For instance, the delivery member 78 can transport the substrates 23 to the third location defined by an opening 83 in the delivery support member 82. Thus, the substrates 23 can travel through the opening 83 toward the conveyor 38. Alternatively, the third location can be configured as a conveyor or other suitable transport member that is configured to transport the substrates 23 toward the conveyor. The third location can be configured as a single opening or conveyor that receives the dosed substrates 23 from all paths 81 or a plurality of the paths 81. Alternatively, each path 81 can have its own dedicated third location.

The dosing machine 72 can include packaging station 42 that delivers a plurality of packages 86 to the conveyor 38. In one example, the packages 86 are placed onto the conveyor 38 upstream of the third location. The dosing machine 72 can include a reservoir that contains a plurality of packages, and can further deliver the packages sequentially onto the conveyor 38. Alternatively, a separate machine can deliver the packages to the conveyor 38. The conveyor 38 causes the packages 86 to move to a position in alignment with a respective one of the third locations. Thus, at least one of the substrates 23 that travels from the delivery member 78 toward the conveyor 38 is delivered into a respective package 86. It is contemplated that in some examples, a single dosed substrate, such as a baked good, will be delivered into each package 86. In other examples, it is contemplated that a plurality of dosed substrates, such as dried fruits and/or nuts, will be delivered into each package 82. For instance, a plurality of the substrates 23 can be delivered from a plurality of the paths 81 up to all of the paths 81 to a single respective container 82. Alternatively, one or more conveyors 38 can deliver the packages to respective locations whereby the packages 82 receive the respective at least one substrate 23 from the respective one of the plurality of paths 81 via the dedicated third location. Thus, packages 86 can thus simultaneously receive the respective at least one substrate 23.

The packaging station 42 can further include a sealing station 88 that is configured to enclose the packages 86 after they have received the respective at least one substrate 23. In particular, the conveyor 38 delivers the packages 86 to the sealing station 88 after they have received the respective at least one substrate 23. The sealing station can seal the package 86 and cause the package 86 to adhere to itself, for instance if the package 86 is a plastic bag or alternatively configured plastic package. The sealing station 88 can alternatively deliver and tighten a cap onto the package 86 if, for instance, the package is configured as ajar or other suitably configured package. The sealed package can then be delivered to a customer

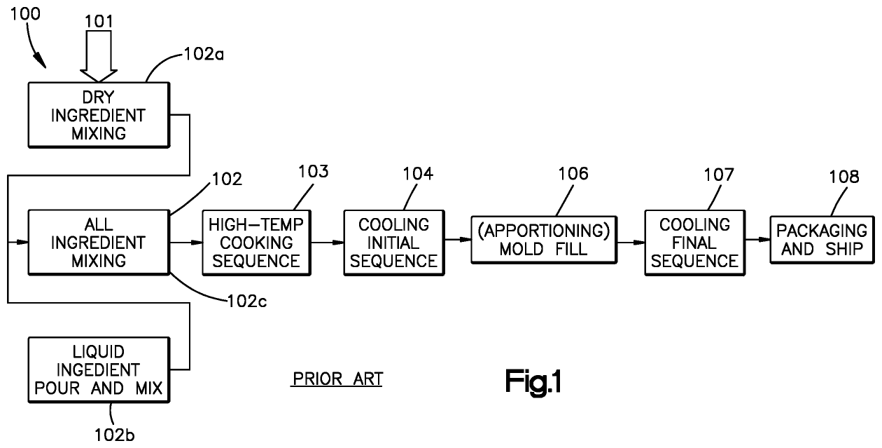

Referring now again to FIG. 1, and as described above, the API can be delivered to food product at any suitable stage during the batch food production process that reduces opportunity for dosage inconsistencies that are common using conventional dosing techniques, and thus produces more consistent API doses in the resulting individualized food products. The present inventors have identified numerous opportunities for errors in conventional API dosing during a method 100 for batch producing cooked food product, which produces inconsistent API dosing in the final food product. To begin, it is recognized that the batch mixing step 102 can include a step 102a of mixing the dry ingredients of the batch food products, and the step 102b of mixing the liquid ingredients of the batch food products, and combining the dry mixed ingredients and the liquid mixed ingredients at step 102. It is recognized that while the steps 102a and 102b are performed separately as shown, they can alternatively be combined into a single mixing step as desired.

As shown in FIG. 1, the mixing step 102 can include a step 102a of mixing together only the ingredients that are in dry form. For a successful product batch, an accurate amount (by volume or weight) is typically desired for each dry ingredient (including the API, if present in the dry ingredients) to be mixed. Deviations from a prescribed volume or weight of each dry ingredient can create an error in the resulting food product. The sum of these individual errors constitutes a total deviation in dry ingredient volume or weight from the prescribed amount. This error will impact the final API concentration of the product batch. In particular, it may shift the API concentration of the entire batch higher or lower with respect to a desired concentration.

The mixing step 102 of FIG. 1 can further include the step 102b of mixing together of only ingredients that are liquids. For a successful product batch, an accurate amount (volume or weight) is typically desired for each liquid ingredient (including the API, if present in the liquid ingredients) to be mixed, and deviations from the prescribed volume or weight of each liquid ingredient can create an error in the resulting food product. The sum of these individual errors constitutes a total deviation in liquid ingredient volume or weight from the prescribed amount. This error will impact the final API concentration of this product batch. In particular, it may shift the API concentration of the entire batch higher or lower with respect to a desired concentration.

The present inventors recognize that errors in the volume of the API in either or both of the dry or liquid mixtures can also linearly impact the resulting API concentration of the resulting food product due to its appearance in the numerator of the concentration calculation: API concentration=API quantity (by weight or volume) divided by the total quantity (by weight or by volume) of all ingredients of the respective mixture. Since there may be several ingredients in the batch recipe, some high deviations may cancel out other low deviations, which can reduce the resultant deviation in the denominator. However, deviations in the quantity of API is not offset by other ingredients, as it is the only quantity in the numerator of the API concentration calculation. Thus any error in the API quantity contributes directly to the resultant error in the API concentration of the batch product and thus of the resulting food product.

It is therefore recognized that potential source of error in the concentration of API in a food product can arise from deviations in the quantity of each ingredient that is to be mixed. Yet another source of error can arise from incomplete mixing of any one up to all of (a) the incomplete mixing together of all the dry ingredients at step 102a, (b) incomplete mixing together of all the liquid ingredients at step 102b, and (c) incomplete mixing together of the dry-ingredient mixture with the liquid-ingredient mixture. In the event that steps 102a and 102b are combined into a single step, then the source or error can arise from incomplete mixing the combination of dry and liquid ingredients. Incomplete mixing can have a direct effect on the concentration of API in the individual food products that are produced from the mixture, as the consistency of the API quantity across the various individual pieces of the product will provide the consumer with a consistent dosage of API in the resulting individual dosed food products. If the API is incompletely combined with the other ingredients of the mixture, then the concentration of API in the resulting individual food products can vary significantly, which will provide the consumer with an API dose that is either too high or too low.

The above-identified errors during the mixing steps can produce deviations in API dose from piece to piece of the resulting food product prior to the cooking step 103. However, still additional opportunities exist for error in the consistency of API in the resulting food product. For instance, inhomogeneities in the dry ingredient mixture, when mixed with the liquids, can cause nodules (also known as “rocks”) of concentrated solid ingredients that persist through the final overall mixing step and absorb more or less than average of the API.

Further still, inhomogeneities can arise is from insufficient duration of mixing at any one up to all of the above-described mixing steps. Mixing can be considered as part art and part technique, as feedback regarding the degree of completion of mixing is often not readily available. Thus, the determination of when to terminate the mixing step can be difficult to specify, and unless the mixing is extensive, inhomogeneity will remain especially in portions of the mixed volume that experience corner or other geometry effects or lack of uniform folding action. These issues can directly impact the ultimate concentration of the API in the resulting individualized food products.

Once the mixing steps are completed, the step 103 of cooking the mixture of batch food product is then performed, such that the food product defines a cooked batch food product. The cooking step 103 can bring the batch food product to a temperature of at least approximately 150 degrees Fahrenheit, such as approximately 200 degrees Fahrenheit, such as from approximately 150 degrees Fahrenheit to approximately 500 degrees Fahrenheit or more, including a range from approximately 250 degrees Fahrenheit to approximately 350 degrees Fahrenheit. Temperature variations throughout the food product, however, can cause varying degrees of decarboxylation of the API. Further, in some instances, the temperatures and time durations required to cook the food product at step 103 can cause the active ingredient(s) of the API to degrade.

With continuing reference to FIG. 1, step 103 represents the high-temperature cooking process (boiling, baking, and the like). In this step the constituents of the batch mixture respond to the significant temperature rise by undergoing a certain amount of evaporation. Different constituents undergo different amounts of evaporation, and the same is true for the constituents of the API such as CBD, THC, terpenes, flavonoids di. Hence the ratios of these API constituents (known as the “profile” of the API) will be altered during the high-temperature cooking. This profile alteration plus the overall reduction in the amount of the API are the two main effects of the high-temperature processing on the final edible product's potency. Furthermore, piece-to-piece variation in this potency reduction will result from any temperature nonuniformity throughout volume of the batch during the cooking. Hence the API overall reduction, the profile modification, and the spatial temperature nonuniformity can all be expected to exacerbate (a) the inaccuracy (deviation from the target dose) of the average API content of the cooked batch, and (b) the inconsistency or variation of the API dose from piece to piece that is experienced by the consumer.

The food product is cooled at step 104, such that the cooked batch food product defines a cooled food product. The cooling step 104 can reduce the temperature of the cooked batch food product by at least 10% of the temperature of the cooked food product, such as at least 20% of the temperature of the cooked food product, including at least 30% of the temperature of the cooked food product. However, inconsistencies of the temperature of the cooled food product can exist as a function of time. Because cooling can cause contraction of the food product, certain regions of the food product can have higher concentrations of API than other regions when the food product is apportioned into individual molds or otherwise individualized at the apportioning step 106, which can provide another source of error in the quantity of API present in the resulting individualized food product. It is appreciated in some examples that the apportioning step 106 can be performed before all cooling sequences in some examples. Thus, step 104 can be performed after step 106.

Once the food product has been packaged, it can then be shipped as desired. As described above, the individualized food products can be further cooled at step 107 prior to packaging. The further cooling step 107 can further decrease the temperature of the food product by at least 10% of the temperature of the cooled food product, such as at least 20% of the temperature of the cooled food product, including at least 30% of the temperature of the cooled food product. It is recognized that the food product can also be passively cooled due to exposure to ambient temperature during the apportioning step 106. Thus, the decreased temperature described above with respect to the further cooling step 107 can apply to the temperature of the food product after the apportioning step 106. However, as described above with respect to the cooling step 104, the step 107 of further cooling can cause further variations in temperature throughout the food product. As a result, when the resulting food product is bigger than a bite-sized edible, the food product can suffer from variations in API concentrations. In particular, a first region of the individualized food product can have a substantially different API concentration than a second region of the individualized food product. It is recognized, of course, that in some examples the step 107 of further cooling can be omitted if the initial cooling step 104 alone sufficiently cooled the food product. FIG. 1 illustrates one examples of a conventional food preparation process, it being recognized that numerous other processes exist depending on the dosed food product being prepared. However, because the food product is dosed prior to the cooking step 103, the resulting food product can suffer from dosing inaccuracies and inconsistencies as described above.

Referring now to FIGS. 7-9, it is recognized that at least one or more up to all of the potential sources of erroring API dosing as described above can be reduced or avoided altogether by performing a step 110 of dosing the food product with API at a different step during the method 100′ for batch producing cooked food product compared to conventional dosing techniques. The method 100′ can include at least some of the food preparation steps of method 100. Thus, the cooking step 103, the cooling step 104, the apportioning or individualizing step 106, the final cooling step 107, and the packaging step 108 of method 100′ can be performed as described above with respect to method 100 with the exception of the step of dosing the API, unless otherwise indicated.

As illustrated in FIG. 7, the method 100′ can include at least the mixing step 102, the cooking step 103, the cooling step 104, and the packaging step 108. As described above, the step 102 of creating a mixture of batch food product provided at step 101, which can include dry ingredients for instance as mixed at step 102a, and wet ingredients, for instance as mixed at step 102b . It is appreciated that step 102 can alternatively include an exclusively wet mixture without dry ingredients, or can alternatively include an exclusively dry mixture without wet ingredients. It should thus be appreciated that at step 102, a mixture of batch food product can be provided by mixing the ingredients of the batch food product. In other examples, the batch food product can be pre-mixed and delivered for use in the subsequent method steps. At step 103, the mixture is cooked (such as baked broiled, boiled, steamed, fried, grilled, or using any suitable alternative cooking method) to create a food product that can define a cooked food product. As described above, the cooking step 103 of method 100′ can increase a temperature of the batch food product to a temperature of at least approximately 200 degrees Fahrenheit, such as from approximately 200 degrees Fahrenheit to approximately 500 degrees Fahrenheit or more, including a range from approximately 250 degrees Fahrenheit to approximately 350 degrees Fahrenheit.

At step 104, the food product can be cooled, such that the food product defines a cooled food product. As described above, the cooling step 104 of method 100′ can reduce the temperature of the cooked batch food product by at least 10% of the temperature of the cooked food product, such as at least 20% of the temperature of the cooked food product, including at least 30% of the temperature of the cooked food product. Eventually, by way of the cooling step 104 or the cooling step 104 in combination with one or more other cooling steps, the temperature of the food product can reach approximately room temperature. The cooling step 104 or any cooling steps described herein can be an active cooling step whereby the temperature of the food product is reduced by actively exposing the food product to a cooling element, such as cooled air, that has a temperature less than room temperature. Alternatively, the cooling step cooling step 104 or any cooling steps described herein can be a passive cooling step whereby the temperature of the food product is reduced by exposing the food product to ambient temperature, which can be room temperature in some examples. Finally, the food product can be packaged and shipped at step 108.

As illustrated at FIG. 7, the method 100′ can include a dosing step 110 that includes delivering the API to the food product after the cooking step 103. For instance, the dosing step 110 can be performed after the cooking step 103 and prior to any cooling step 104. Alternatively, the dosing step 110 can be applied after the cooking step 103 and after the cooling step 104. Thus, the entire batch of food product can be dosed with the API. Because the API is delivered to the food product after the mixing step 102, the API is not subject to variations throughout the mixture due to imperfections during the mixing process as is the case during the conventional method 100 of FIG. 1. Further, because the API can also be delivered to the food product after the cooking step 103 in one example, the API is not subject to degradation during the cooking step 103 as is the case during the conventional method 100 of FIG. 1. It should be appreciated that the API delivered to the food product at step 110 in all examples of FIGS. 7-11 described herein can be disposed in the resulting final cooled food product unless otherwise indicated.

Further, it is recognized that if the API such as a cannabinoid is not decarboxylated, and thus is in its acidic form, it can remain in the acidic form when dosed after the cooking step 103. In contrast, in the conventional method 100, API ingredient that is not initially decarboxylated can become decarboxylated during the cooking step 103. Thus, in method 100′ the API can be dosed in its acidic form (i.e., not decarboxylated) and can remain in its acidic form if desired. Without being bound by theory, it is believed that the acidic form of the API can possess certain medical benefits when dosed and maintained and consumed in its acidic form. It is appreciated, of course, that the dosed API can be provided in its decarboxylated (e.g., non-acidic or neutral) form if desired, and subsequently delivered to the food product in its decarboxylated form. It should be appreciated in some examples that the API can be partially decarboxylated when delivered to the food product. However, in these examples, the API is not fully decarboxylated. Thus, the API is more acidic than those APIs that are fully decarboxylated using conventional batch dosing methods.

Alternatively or additionally, the API can be delivered to the food product at dosing step 110 after the cooking step 103, and after some amount of cooling of the food product (or no amount of cooling depending on the cooking temperature) such that the temperature of the food product does not cause the API to become fully decarboxylated during the dosing step 110. Thus, the API can become at least partially non-decarboxylated. As described above, the API can be a cannabinoid in one example. During the method, the API can be dosed such that it becomes up to approximately 80% decarboxylated, and thus can be at least approximately 20% non-decarboxylated. For instance, the API can become up to approximately 70% decarboxylated, and thus can be at least approximately 30% non-decarboxylated. For instance, the API can become up to approximately 60% decarboxylated, and thus can be at least approximately 40% non-decarboxylated. For instance, the API can become up to approximately 50% decarboxylated, and thus can be at least approximately 50% non-decarboxylated. For instance, the API can become up to approximately 40% decarboxylated, and thus can be at least approximately 60% non-decarboxylated. For instance, the API can become up to approximately 30% decarboxylated, and thus can be at least approximately 70% non-decarboxylated. For instance, the API can become up to approximately 20% decarboxylated, and thus can be at least approximately 80% non-decarboxylated. For instance, the API can become up to approximately 10% decarboxylated, and thus can be at least approximately 90% non-decarboxylated. In still other examples, the API can remain completely non-decarboxylated. Once the API has been delivered to the food product at dosing step 110, the food product can be sufficiently viscous so as to be deliverable into individual molds, as discussed in more detail below.

In some examples, the API can be delivered to the food product at dosing step 110 after the cooking step 103, and after some amount of cooling of the food product (or no amount of cooling depending on the cooking temperature) such that the temperature of the food product does not cause the API to become fully evaporated. If desired, the quantity of API to added to the food product can be determined based, in part, on the quantity of API that will become evaporated due to the temperature of the food product during the dosing step 110. In one example, up to approximately 80% of the API by weight can be evaporated. For instance, up to approximately 70% of the API by weight can be evaporated. For instance, up to approximately 60% of the API by weight can be evaporated. For instance, up to approximately 50% of the API by weight can be evaporated. For instance, up to approximately 40% of the API by weight can be evaporated. For instance, up to approximately 30% of the API by weight can be evaporated. For instance, up to approximately 20% of the API by weight can be evaporated. For instance, up to approximately 10% of the API by weight can be evaporated. In still other examples, none of the API is evaporated. As described above, once the API has been delivered to the food product at dosing step 110, the food product can be sufficiently viscous so as to be deliverable into individual molds, as discussed in more detail below.

As will now be described with respect to FIG. 8, the method 100′ can include the step of delivering the API to individualized food product. In particular, the batch food product can be apportioned into individualized food products. Thus, after the individualizing step, the food product can be said to define individualized food products. The API can then be delivered to the individualized food products in accordance with any suitable delivery method described herein, including the delivery of microdroplets. In one example, the individualizing step can occur at step 106, whereby the food product is apportioned into individual molds, such that the cooled food product is apportioned into individualized food products. For instance, the dosing step 110 can be performed after the cooled food product has been delivered to the respective molds at step 106. As a result, the substrate to be dosed in the manner described above can be configured as one or more individualized food products produced at step 106. Thus, the dosing step 110 can be performed prior to the step of removing the individualized food products from the respective molds and packaging the individualized food products at step 108.

The dosing step 110 can include one or more up to all of 1) delivering the API such as cannabinoid to the molds prior to the step of delivering the food product into respective molds, such that the cooled food product contacts the delivered cannabinoid in the respective molds during the step of delivering the food product into respective molds, 2) delivering the cannabinoid directly to the individualized food products while the individualized food products are disposed in the respective molds, and 3) delivering the cannabinoid directly to the cooled food product during delivery of the food product into the respective molds. In one example, the step of delivering the cannabinoid and food products to the respective molds can include the step of alternatingly adding layers of edible food product and cannabinoid, such that cannabinoid is disposed between adjacent layers of food product.

The food product can be considered individualized food products as the food product is delivered into the respective molds. In some examples, such as when the food product is configured as a gummy candy, the cooled food product can be viscous and flowable, and can be driven to flow into the respective molds. The individualized food products can define cooled individualized food products if delivered to the molds after the cooling step 104. In some examples, it is recognized that the cooked food product can be delivered to the molds at step 106 after the cooking step 103 and prior to the cooling step 104 if desired. Thus, the step 110 of dosing the individualized food product apportioned into the molds can be performed after the cooking step 103 and prior to the cooling step 104. The individualized food products can thus define cooked individualized food products after the cooking step 103 and prior to the cooling step 104.

Thus, it is recognized that the API can be delivered to the individualized food product after it has been cooked at step 103, in the manner described above with respect to FIGS. 7-8. In one example, the API can be delivered to cooked food product that has been individualized in respective molds. In another example, for instance when the food product is configured as a baked good, the cooked food product can be non-viscous, and the individualized food products can be physically divided or apportioned, such as during a cutting operation, from a remainder of the cooked food product. The API can thus be delivered to the batch food product after the cooking step (and before or after the cooling step 104) and before dividing or apportioning the food product into individualized food products. Alternatively, the API can be delivered to the batch food product after the cooking step (and before or after the cooling step 104) and after dividing or apportioning the food product into individualized food products.

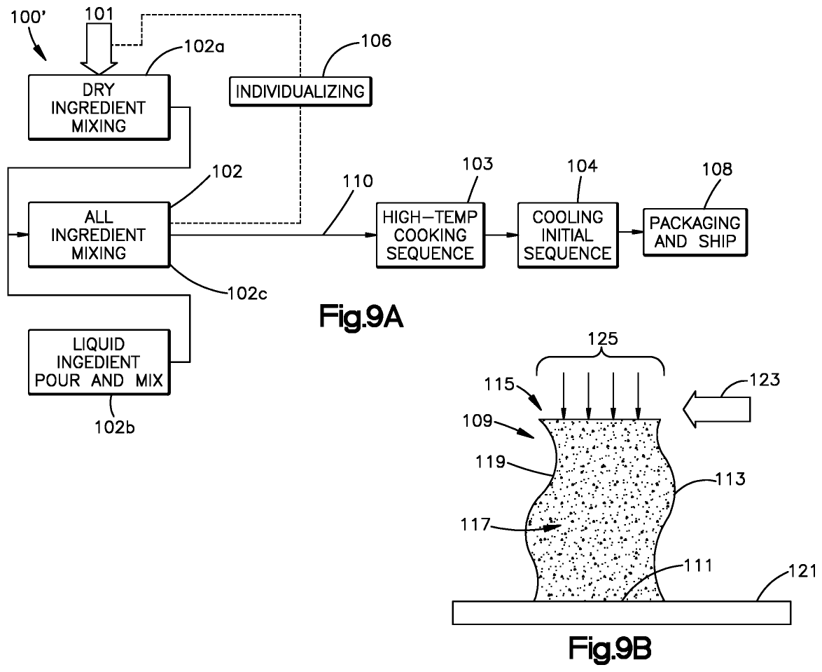

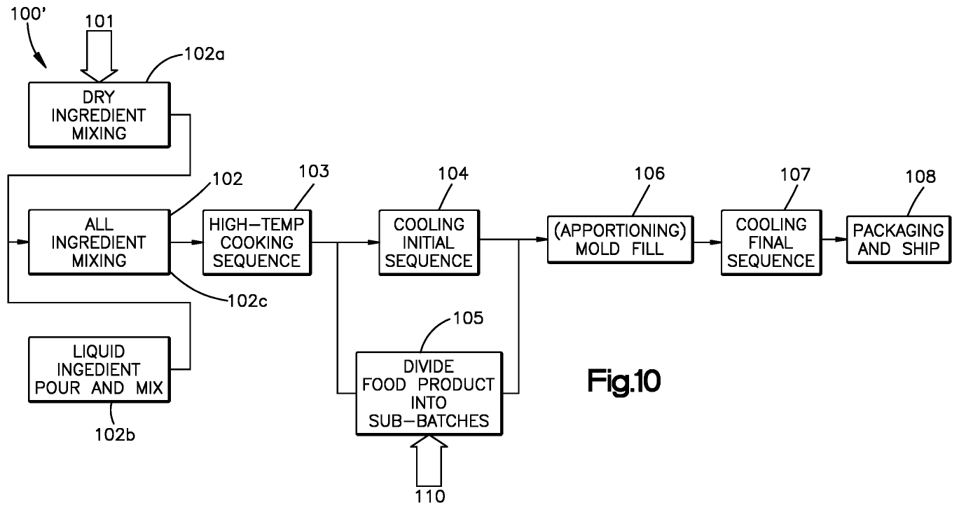

Alternatively, referring now to FIG. 9A, it is appreciated that the method 100′ can realize certain benefits with respect to the conventional method 100 of FIG. 1 when the food product is individualized prior to the cooking step 103. In particular, dosing the individualized food product prior to the cooking step 103 exposes the individualized food product to less variation due to potentially imperfect mixing than would occur in a batch mixture. Further, even if the individualized food product is imperfectly mixed at step 102 of FIG. 9A, independent of that the API can still precisely be delivered as microdroplets to the food product. Thus, the imperfect mixing has a minimal or no impact on the dose of the API delivered to the individualized food product. The API can be delivered to the individualized food product either before or after the cooking step 103, as described above

Thus, in one example shown in FIG. 9A, the food product can be individualized at the step 101 of providing ingredients of a food product to be prepared. In particular, the ingredients can be apportioned into individual molds prior to being mixed at step 102 to produce individualized food product mixtures. The API can be delivered at step 110 to each of the individualized food product mixtures as described above to define an API-containing or dosed individualized food product mixtures. In some examples, the API such as cannabinoid can be delivered to the molds prior to the step of delivering the food product ingredients into respective molds. Alternatively, the food product ingredients can be delivered to the molds, and the API can be subsequently delivered to the individualized food product ingredients while the individualized food product mixtures are disposed in the respective molds. Alternatively still, the API can be delivered into the molds while the food product ingredients are being delivered into the molds.

In another example shown in FIG. 9A, the food product can be individualized after completion of the step 102 of mixing the food product. In particular, the ingredients can be provided at step 101 and mixed as a batch at step 102. Subsequently, the mixed batch food product prepared at step 102 can be individualized to produce individualized food product mixtures. The API can be delivered to each of the individualized food product mixtures (again as step 110 in FIG. 9A) as described above, such that the food product defines dosed individualized food product mixtures. In some examples, the API such as cannabinoid can be delivered to the molds prior to the step of delivering the food product mixture into respective molds. Alternatively, the food product mixture can be delivered to the molds, and the API can be subsequently delivered to the individualized food product mixture while the individualized food product mixture is disposed in the respective molds. Alternatively still, the API can be delivered into the molds while the food product mixtures are being delivered into the molds. For instance, API can be delivered into the molds between successive layers of mixture that are added to the molds. Thus, the API can be disposed between successive layers of the mixture.

Referring to FIG. 9B, each mold 109 can each define a base 111, and at least one side wall 113 supported by the base 111 so as to define an internal mold cavity 117 that receives the food product mixture 119. Some of the molds 109 can share a common side wall 113. The base 111 can define a closed lower end of the mold cavity 117, and the at least one side wall 113 can define an open end 115 that is configured to receive the food product. The base 111 and the at least one side wall 113 can define any suitable size and shape as desired. In some examples, the side wall 113 can be rigid. In other examples, the side wall 113 can be flexible so as to pulsate, thereby further mixing the food product mixture 119, with or without the API, disposed in the mold cavity 117. Alternatively or additionally, the mold 109 can be disposed on a support surface 121 that can be configured to vibrate, thereby agitating and further mixing the food product mixture 119 disposed in the mold cavity 117. Alternatively or additionally still, airflow 123 can be directed across the exposed surface of the food product mixture 119 so as to stimulate agitation of the food product mixture 119. Alternatively or additionally still, energy 125 can be directed into the food product mixture 119. The energy can be configured as a propagating electromagnetic wave or illumination (infrared, ultrasound, microwave) pulses.

The dosed individualized food product mixtures can then be cooked at step 103, cooled at step 104, and packaged at step 108 as desired. It is further appreciated that API can also, if desired, be delivered to the food product after the cooking step in the manner described herein. Alternatively still, the individualized food product mixtures can be cooked without first being dosed with API, and then can be dosed with API after the cooking step 103 and/or the cooling step 104, but before the packaging step 108 in the manner described above.

The steps of delivering the API can be achieved by delivering microdroplets of API in the manner described above. Thus, the dosing step 110 can include one or more up to all of 1) delivering microdroplets of the API to the molds prior to the step of delivering the food product into respective molds, such that the food product contacts the delivered cannabinoid in the respective molds during the step of delivering the food product into respective molds, 2) delivering microdroplets of the cannabinoid directly to the food product as the food product is delivered into the respective molds, and 3) delivering microdroplets of the cannabinoid directly to the individualized food products while the individualized food products are disposed in the respective molds. The microdroplets can be delivered in accordance with any example described herein.

Thus, the dosing step 110 can include the step of delivering at least one microdroplet of the API-containing liquid from the dosing station to the individualized food products in the manner described above. Similarly, the dosing step can include the step of bringing the individualized food products into alignment with a dosing head of the dosing station, and the step of delivering at least one microdroplet comprises delivering the at least one microdroplet from the dosing head to the individualized food products. In this regard, the dosing step can include delivering respective APIs to different ones of the individualized food products, wherein the respective APIs have at least one API characteristic different from each other. The different API characteristic comprises at least one of 1) a concentration of the API, 2) a volume of API delivered to the individualized food products during the dosing step, 3) a composition of the API, 4) a modifier mixed with the API, the modifier configured to modify at least one of a flavor, a mechanical property, and an aesthetic of the delivered API, and 5) a location of at least one dosing zone of the individualized food products. Alternatively or additionally, the step of delivering at least one microdroplet of the API-containing liquid can include delivering a different API to different subzones in a delivery zone of the individualized food products. Further, the method 100′ can include the step of, after the dosing, post-processing the individualized food products. As described above, the post-processing can at least one of 1) dry the solvent, 2) increase the viscosity of the API, 3) further adheres the API to the individualized food product, 4) disperse the API along the individualized food product, and 5) increase absorption of the API into the individualized food products.

Referring now to FIG. 10, it is recognized that, in some methods 100′, it may be desirable to divide the batch food product into more manageable sub-batches that have a quantity of food product less than that of the batch food product but more than that of an individual food product. One or more of the sub-batches can then be dosed with the API at step 110. The step 105 of dividing the cooked batch food product to produce at least one sub-batch such as a plurality of sub-batches can be performed before the cooling step 104. Alternatively, the step of dividing the cooked batch food product to produce at least one sub-batch such as a plurality of sub-batches can be performed after the cooling step 104. The step 105 of producing the sub-batches can occur before the dosing step 110. The step of dividing the cooked batch food product to produce at least one sub-batch can include the step of delivering the cooked product into at least one container to produce the at least one sub-batch. For instance, each sub-batch can be delivered into a separate distinct container. Alternatively, more than one sub-batch can be delivered into a single container that has separate compartments. It should be appreciated that the cooked batch food product that is divided into sub-batches can also be configured as the cooled batch food product that was subjected to the cooling step 104 prior to being divided into sub-batches.

The sub-batches can be dosed in any manner as described above with respect to the individualized food product. Thus, in one example, the dosing step can include one or more up to all of 1) delivering the API to the at least one container prior to the step of dividing the cooked food product into respective at least one container so as to define the at least one sub-batch, such that the at least one sub-batch contacts the delivered API in the at least one container during the step of dividing the cooked batch food product to produce at least one sub-batch, 2) delivering the API directly to the cooked food product during the step of dividing the cooked batch food product to produce at least one sub-batch (i.e., as the cooked food product is delivered into the respective at least one container), and 3) delivering the API directly to the at least one sub-batch after the cooked batch food product has been divided into sub-batches (i.e., after the cooked food product has been delivered to the at least one container. In this regard, it is appreciated that the cooked food product can be considered a sub-batch as the cooked food product is delivered into the respective molds. Because the sub-batches include a greater quantity of food product than is intended for individualized food product, the sub-batches can be apportioned into individualized food products in any manner as desired at apportioning step 106 after they have been dosed with API. A final cooling step can be performed at step 107 as described above, if desired, and the individualized food product can be packaged at step 108. The final cooling step 107 can be performed before or after the apportioning step 106.

In another example, referring now to FIG. 11, it is appreciated that the method 100′ can realize certain benefits with respect to the conventional method 100 of FIG. 1 when the mixed food product is divided into mixed sub-batches prior to the cooking step 103, and the API is delivered to the mixed sub-batches. In particular, dosing the mixed sub-batches prior to the cooking step 103 exposes the food product of the sub-batches to less variation due to potentially imperfect mixing than would occur in a batch mixture. Further, even if the sub-batches are imperfectly mixed, the API can be delivered as microdroplets to the sub-batches. Thus, the imperfect mixing has a minimal or no impact on the dose of the API delivered to the individualized food product. It is appreciated that the API can be delivered to the sub-batches either before or after the cooking step 103.

Thus, in one example shown in FIG. 11, the sub-batches can be provided after the step 101 of providing ingredients of a food product to be prepared. In particular, the ingredients can be provided and combined in batch quantity, and mixed at step 102. Subsequently, the mixed food product prepared at step 102 can be divided into sub-batches at step 105. Next, the API can be delivered to each of the sub-batches as described above, such that the mixed sub-batches define dosed mixed sub-batches. The dosed mixed sub-batches can then be cooked, cooled, and as desired. Alternatively, the mixed sub-batches can be cooked at step 103 to produce cooked sub-batches, and the API can then be delivered to the cooked sub-batches after the cooking step 103. For instance, the API can be delivered to the cooked sub-batches after the cooling step 104 if desired, but before the packaging step. As described above, the sub-batches of food product can be apportioned into individualized food product at step 106. The individualized food product can be subject to a final cooling sequence 107, if desired, and the individualized food product can be packaged at step 108.

The steps of delivering the API to the sub-batches can be achieved by delivering microdroplets of API in the manner described above. Thus, the dosing step 110 can include one or more up to all of 1) delivering microdroplets of the API to the at least one container prior to the step of dividing the food product into respective containers so as to define the sub-batches. Thus, the sub-batches contact and adhere to the delivered API in the at least one container during the step of dividing the cooked batch food product to produce at least one sub-batch, 2) delivering microdroplets of the API directly to the food product during the step of dividing the cooked batch food product to produce the sub-batches (i.e., as the cooked food product is delivered into the respective at least one container), and 3) delivering microdroplets of the API directly to the at least one sub-batch after the food product has been divided into sub-batches (i.e., after the food product has been delivered to the at least one container). The microdroplets can be delivered in accordance with any example described herein.

Thus, the dosing step 110 can include the step of delivering at least one microdroplet of the API-containing liquid from the dosing station to the sub-batches of food product in the manner described above. Similarly, the dosing step can include the step of bringing the sub-batches to be dosed into alignment with a dosing head of the dosing station, and the step of delivering at least one microdroplet can include delivering the at least one microdroplet from the dosing head to the sub-batch. In this regard, the dosing step can include delivering respective APIs to different ones of the sub-batches, wherein the respective APIs have at least one API characteristic different from each other. The different API characteristic comprises at least one of 1) a concentration of the API, 2) a volume of API delivered to the sub-batch during the dosing step, 3) a composition of the API, 4) a modifier mixed with the API, the modifier configured to modify at least one of a flavor, a mechanical property, and an aesthetic of the delivered API, and 5) a location of at least one dosing zone of the sub-batch. Alternatively or additionally, the step of delivering at least one microdroplet of the API-containing liquid can include delivering a different API to different subzones in a delivery zone of the sub-batch. Further, the method 100′ can include the step of, after the dosing, post-processing the dosed sub-batches. As described above, the post-processing can at least one of 1) dry the solvent, 2) increase the viscosity of the API, 3) further adheres the API to the sub-batches, 4) disperse the API along the sub-batches, and 5) increase absorption of the API into the sub-batches. In some examples, such as when the food product is configured as a gummy candy, the cooked food product can be viscous and can flow into the respective at least one container so as to produce the sub-batch. In other examples, such as when the food product is configured as a baked good, the cooked food product can be non-viscous, and can be physically divided such as cut from a remainder of the cooked food product and delivered to the container.

It should be further appreciated that the food product of the sub-batches can be further individualized as desired as described above with respect to step 106. Further, the individualized food product can be dosed with API as desired, in the manner described above. Thus, when the food product is divided into sub-batches, the food product can be dosed with the API as described above either or both of 1) when the food product has been divided into sub-batches, and 2) when the food product has been individualized. Thus, in one example, a first portion of a desired dose of API can be delivered to the sub-batches, and a second portion of a desired dose of API can be delivered to the individualized food product. Therefore, dividing the food product into sub-batches can provide a two-stage API delivery system wherein a first quantity of API is dosed, and then in the later step after apportioning a second quantity of API is dosed. The second quantity of API can be configured for rapid sublingual uptake upon ingestion of the food product. The first quantity of API can enter the bloodstream upon digestion of the food product. In some examples, the first quantity of API can be greater than the second quantity of API.

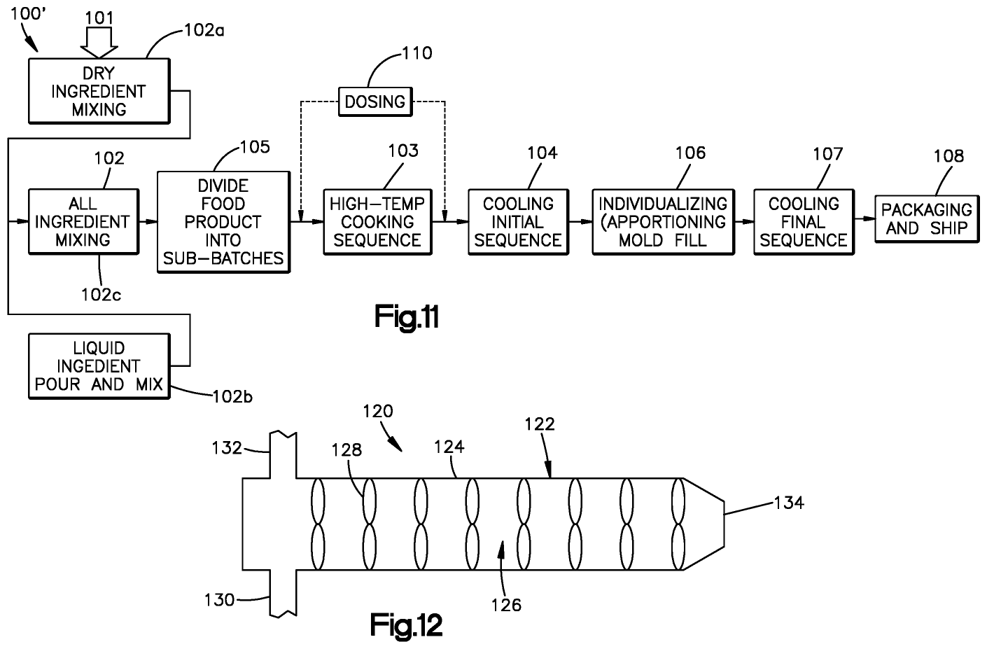

Referring now to FIGS. 9 and 11-12, examples have been provided that disclose methods for dosing a food product with API prior to the cooking step 103. While certain examples of methods have been disclosed for mixing the API with the ingredients prior to the cooking step 103, it should be appreciated that the API can be mixed with the ingredients in accordance with any suitable alternative example as desired. For example, at least one mixing device 120 can be configured to mix the API with the ingredients. In one example, the mixing device 120 can be configured as a mixing tube 122. The mixing device 120 can define an elongate body 124 that defines a mixing chamber 126, and at least one mixing element 128 in the mixing chamber that is configured to mix food product ingredients and AIP that are disposed in the mixing chamber 126. The body 124, and thus the mixing chamber 126, can be substantially cylindrical in shape, substantially conical in shape, or can define any suitable alternative shape as desired. The mixing element 128 can be configured as an auger, one or more mixing blades, or the like, that can be actuated to operate by rotating in the mixing chamber 126 so as to mix ingredients that are disposed in the mixing chamber 126. The mixing device 120 can include at least one food product input conduit 130 that extends through the body 124 and is configured to deliver one or more ingredients of the batch food product into the mixing chamber 126, and at least one API input conduit 132 that extends through the body 124 and is configured to deliver at least one API into the mixing chamber 126.

For instance, the at least one dry ingredient of the food product and the at least one wet ingredient of the food product can be delivered into the mixing chamber 126 through at least one food product input conduit 130. In one example, the dry and wet ingredients of the food product can be delivered into the mixing chamber 126 through the same product input conduit 130. Alternatively, different groupings of one or more dry ingredients can be introduced into the mixing chamber through respective different dry food product input conduits 130. Similarly, different groupings of one or more wet ingredients can be introduced into the mixing chamber through respective different wet food product input conduits 130. It should be appreciated that some or all of the food product ingredients can be pre-mixed, for instance in a mixing tube, prior to being introduced into the mixing chamber 126 through the respective product input conduit 130. Alternatively,

[symbol]

The API can be delivered to the mixing chamber 126 to be mixed with the food product ingredients. In one example, the API is delivered to the mixing chamber 126, for instance through the API input conduit 132. For instance, a predetermined quantity of API can be poured, pipetted, delivered as discrete microdroplets as described herein, or delivered into the mixing chamber 126 in any manner as desired. One or more APIs can be delivered through a single API input conduit 132. Alternatively, multiple APIs can be delivered through respective different API input conduit 132. Thus, it can be said that at least one API can be delivered into the mixing chamber 126 through at least one API input conduit 132. In one example, the API can be delivered into the mixing chamber 126 prior to the delivery of food product ingredients into the mixing chamber 126. Alternatively, the API can be delivered into the mixing chamber 126 after to the delivery of food product ingredients into the mixing chamber 126. Alternatively still, the API can be delivered into the mixing chamber 126 during the delivery of food product ingredients into the mixing chamber 126.

The dosed food product mixture can then be heated as step 103. For instance, the mixing device 120 can define an outlet 134 that is configured to deliver the mixture from the mixing device 120 to any suitable carrier to deliver the mixture to an oven that performs the cooking step 103. The cooked food product can then be divided into individualized food product and packaged in the manner described above, or can be delivered to individual molds at step 106 as described above. In other examples, the API-containing mixture can be delivered from the mixing device 120 directly to individualized molds, or to an intermediate carrier that delivers the API-containing mixture to the individualized molds. The molds can then be heated at step 103 as desired, and allowed to cool before being packaged.

As described above, the mixing device 120 can include at least one mixing element 128 in the mixing chamber that is configured to mix food product ingredients and AIP that are disposed in the mixing chamber 126 to create the API-containing mixture. The mixing element 128 can be actuated as and/or after the ingredients and API are introduced into the mixing chamber 126, thereby creating the API-containing mixture. As described above, the body 124, and thus the mixing chamber 126 can be substantially conical in shape. Thus, the mixing chamber 126 can define a vortex during operation of the mixing elements 128 that both mixes the food product ingredients and API, and further drives the resulting mixture through the outlet 134.

As described above, the method 100′ for batch producing cooked food product can be applied to any food product as desired that is the product of mixing ingredients and heat. By way of example and not limitation, the food product can be a candy or a baked good, or any alternative food product as desired. In one example, the candy can be a gummy candy or a hard candy as desired. The individual candies can be individually wrapped. Alternatively, unwrapped candies can be placed inside a single package. Alternatively still, the individually wrapped candies can be placed inside a single package. The candies can be bite-sized, meaning sized to be placed into the mouth of a consumer whole, or can be sized to be consumed in iterative bites. When the food product is a baked good, the baked good can be individually wrapped. Alternatively, more than one brownie can be placed in a common container. Alternatively still, individually wrapped baked goods can be placed in a common container. The baked goods can be sized to be placed into the mouth of a consumer whole, or can be sized to be consumed in iterative bites.

It should be noted that the illustrations and discussions of the embodiments and examples shown in the figures are for exemplary purposes only and should not be construed limiting the disclosure. One skilled in the art will appreciate that the present disclosure contemplates a range of possible modifications of the various aspects, embodiments and examples described herein. Additionally, it should be understood that the concepts described above with the above-described embodiments and examples may be employed alone or in combination with any of the other embodiments and examples described above. It should further be appreciated that the various alternatives described above with respect to one illustrated embodiment can apply to all other embodiments and examples described herein, unless otherwise indicated. Reference is therefore made to the claims.

What is claimed:

1. A method of preparing a food product comprising the steps of:
providing a mixture of food product to define a mixture of batch food product;
cooking the mixture of batch food product such that the food product defines a cooked food product;
cooling the cooked food product, such that the food product defines a cooled food product; and
after the providing step, dosing the food product with an active pharmaceutical ingredient (API).

2. The method of claim 1, wherein the dosing step is performed after the cooking step.

3. The method of claim 2, wherein the API comprises a cannabinoid.

4. The method of claim 2, wherein the dosing step is performed prior to the cooling step.

5. The method of claim 1, wherein the dosing step is performed after the cooling step.

6. The method of claim 1, wherein the API comprises a cannabinoid.

7. The method of claim 1, further comprising the step of individualizing the food product to define individualized food products.

8. The method of claim 7, wherein the individualizing step is performed after the cooking step and before the cooling step.

9. The method of claim 7, wherein the individualizing step is performed after the cooling step.

10. The method of claim 7, wherein the individualizing step is performed after the mixing step and prior to the cooking step.

11. The method of claim 31, wherein the individualizing step comprises delivering the food product into respective molds, such that the food product is apportioned into individualized food products.

12. The method of claim 31, wherein the dosing step comprises dosing the individualized food products with the API.

13. The method of claim 11, wherein the dosing step comprises delivering the API to the molds prior to the step of delivering the cooled food product into respective molds, such that the cooled food product contacts the delivered API in the respective molds during the step of delivering the cooled food product into respective molds.

14. The method of claim 11, wherein the dosing step comprises delivering the API directly to the cooled food product as the cooled food product is delivered into the respective molds.

15. The method of claim 14, wherein the dosing step comprises delivering the cannabis directly to the individualized food products while the individualized food products are disposed in the respective molds.

16. The method of claim 15, wherein the API is disposed between successive layers of the individualized food product in the respective molds.

17. The method of claim 11, further comprising the step of packaging the individualized food products.

18. The method of claim 11, comprising the step of further cooling the individualized food products after the cooling step has been performed.

19. The method of claim 18, wherein the step of further cooling is performed after the dosing step.

20. The method of claim 1, further comprising the step of delivering the mixture to a plurality of molds.

21. The method of claim 20, further comprising the step of stimulating agitation of the mixture after the step of dosing the food product with the active pharmaceutical ingredient (API).

22. The method of claim 1, wherein the providing step comprises delivering ingredients of the mixture into a mixing chamber of an apparatus, and the dosing step comprises delivering API into the mixing chamber, the method further comprising the step of mixing the ingredients and the cannabis so as to define a dosed mixture.

23. The method of claim 1, further comprising the step of dividing the food product into at least one sub-batch, such that the dosing step comprises dosing the at least one sub-batch with the API.

24. The method of claim 1, wherein the API comprises an extract from a marijuana plant, hemp plant.

25. The method of claim 24, wherein the API comprises one or both of a flavonoid and a terpene.

26. The method of claim 1, wherein the API comprises one or both of a flavonoid and a terpene.

27. The method of claim 1, wherein the API comprises a nutraceutical.

28. The method of claim 1, wherein the food product is a baked good.

29. The method of claim 1, wherein the food product is a gummy candy.
